(19) United States
(12) Patent Application Publication
(10) Pub. No.: US 2015/0150156 A1
(43) Pub. Date: May 28, 2015

(54) METHODS AND COMPOSITIONS FOR REDUCING GENE EXPRESSION IN PLANTS

(71) Applicant: The Regents of the University of California, Oakland, CA (US)

(72) Inventors: Steve E. Jacobsen, Agoura Hills, CA (US); Elena Caro, Madrid (ES)

(21) Appl. No.: 14/371,986

(22) PCT Filed: Jan. 11, 2013
(86) PCT No.: PCT/US13/21310

§ 371 Date: Jul. 11, 2014

Related U.S. Application Data
(60) Provisional application No. 61/585,619, filed on Jan. 11, 2012.

(51) Int. Cl. C12N 15/82; C12N 9/10

(52) U.S. Cl. CPC C12N 15/8218; C12N 9/1007; C12Y 201/01043
USPC 800/278; 536/23.2

(57) ABSTRACT

The present disclosure relates to recombinant methyltransferases that epigenetically silence gene expression and to methods of using such proteins for reducing the expression of genes in plants.

### CROSS-REFERENCE TO RELATED APPLICATIONS

This application claims the benefit of U.S. Provisional Application No. 61/585,619, filed on Jan. 11, 2012, which is incorporated by reference herein in its entirety.

### STATEMENT REGARDING FEDERALLY-SPONSORED RESEARCH

This invention was made with government support under grant GM060398 awarded by the National Institutes of Health (NIH). The government has certain rights in the invention.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 262232000240SEQLIST.txt, date recorded: Dec. 20, 2012, size: 326 KB).

### FIELD

The present disclosure relates to recombinant methyltransferases that epigenetically silence gene expression and to methods of using such proteins for reducing the expression of genes in plants.

### BACKGROUND

Epigenetic marks are enzyme-mediated chemical modifications of DNA and of its associated chromatin proteins. Although epigenetic marks do not alter the primary sequence of DNA, they do contain heritable information and play key roles in regulating genome function. Such modifications, including cytosine methylation, posttranslational modifications of histone tails and the histone core, and the positioning of nucleosomes (histone octamers wrapped with DNA), influence the transcriptional state and other functional aspects of chromatin. For example, methylation of DNA and certain residues on the histone H3 N-terminal tail, such as H3 lysine 9 (H3K9), are important for transcriptional gene silencing and the formation of heterochromatin. Such marks are essential for the silencing of nongenic sequences, including transposons, pseudogenes, repetitive sequences, and integrated viruses, that become deleterious to cells if expressed and hence activated. Epigenetic gene silencing is also important in developmental phenomena such as imprinting in both plants and mammals, as well as in cell differentiation and reprogramming.

Different pathways involved in epigenetic silencing have been previously described, and include histone deacetylation, H3K27 and H3K9 methylation, H3K4 demethylation, and DNA methylation of promoters. In plants, no proteins have been described that link the recognition of a specific DNA sequence with the establishment of an epigenetic state. Thus, plant epigenetic regulators generally cannot be used for epigenetic silencing of specific genes or transgenes in plants.

One solution is to identify or engineer epigenetic regulators that contain sequence-specific zinc finger domains, since zinc fingers were first identified as DNA-binding motifs (Miller et al., 1985), and numerous other variations of them have been characterized. Recent progress has been made that allows the engineering of DNA-binding proteins that specifically recognize any desired DNA sequence. For example, it was recently shown that a three-finger zinc finger protein could be constructed to block the expression of a human oncogene that was transformed into a mouse cell line (Choo and Klug, 1994). However, potential problems to engineering epigenetic regulators that contain an engineered zinc finger domain include ensuring that the engineered protein will have the correct folding to be functional, and ensuring that the fusion of the zinc finger domain to the epigenetic regulator does not interfere with either the DNA-specific binding of the zinc finger domain or the activity of the epigenetic regulator.

Accordingly, a need exists for improved epigenetic regulators, such as methyltransferases, that are capable of binding specific DNA sequences, that fold properly, and that retain both the sequence-specific DNA-binding activity and epigenetic gene silencing activity when expressed in plants.

### BRIEF SUMMARY

In order to meet the above needs, the present disclosure provides novel recombinant SUVR5 proteins that contain a DNA-binding domain, a C-terminal pre-SET domain, a C-terminal SET domain, and a C-terminal post-SET domain; and methods of using such recombinant SUVR5 proteins for reducing expression of one or more target nucleic acids, such as genes, in a plant.

Accordingly, certain aspects of the present disclosure relate to a method for reducing expression of one or more target nucleic acids in a plant, by (a) providing a plant containing a recombinant polypeptide, where the recombinant polypeptide contains a DNA-binding domain, a C-terminal pre-SET domain, a C-terminal SET domain, and a C-terminal post-SET domain; and (b) growing the plant under conditions whereby the recombinant polypeptide binds to the one or more target nucleic acids, thereby reducing expression of the one or more target nucleic acids.

In certain embodiments, the DNA-binding domain contains a zinc finger domain. In certain embodiments, the zinc finger domain contains two, three, four, five, six, seven, eight, or nine zinc fingers. In certain embodiments, the zinc finger domain is a zinc finger array. In certain embodiments, the zinc finger domain is selected from a Cys2His2 (C2H2) zinc finger domain, a CCCH zinc finger domain, a multi-cysteine zinc finger domain, and a zinc binuclear cluster domain. In certain embodiments, the DNA-binding domain is selected from a TAL effector targeting domain, a helix-turn-helix family DNA-binding domain, a basic domain, a ribbon-helix-helix domain, a TBP domain, a barrel dimer domain, a real homology domain, a BAH domain, a SANT domain, a Chromodomain, a Tudor domain, a Bromodomain, a PHD domain, a WD40 domain, and a MBD domain. In certain embodiments, the DNA-binding domain contains a TAL effector targeting domain. In certain embodiments, the DNA-binding domain contains three C2H2 zinc finger domains. In certain embodiments, the DNA-binding domain contains an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1. In certain embodiments, the DNA-binding domain contains an amino acid sequence 100% identical to SEQ ID NO: 1.

In certain embodiments that may be combined with any of the preceding embodiments, the recombinant polypeptide further contains one or more additional DNA-binding domains. In certain embodiments, the one or more additional DNA-binding domains contain an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1. In certain embodiments, the one or more additional DNA-binding domains contain an amino acid sequence 100% identical to SEQ ID NO: 1. In certain embodiments, the one or more additional DNA-binding domains contain an amino acid sequence heterologous to SEQ ID NO: 1.

In certain embodiments that may be combined with any of the preceding embodiments, the C-terminal pre-SET domain contains an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 2 or SEQ ID NO: 3. In certain embodiments that may be combined with any of the preceding embodiments, the C-terminal SET domain contains an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to an amino acid sequence selected from SEQ ID NOs: 4, 5, and 6. In certain embodiments that may be combined with any of the preceding embodiments, the C-terminal post-SET domain contains an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 7. In certain embodiments, the recombinant polypeptide contains an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 8.

In certain embodiments that may be combined with any of the preceding embodiments, the recombinant polypeptide complexes with one or more polypeptides selected from a DNA methyltransferase, a histone methyltransferase, a histone deacetylase, a histone demethylase, a chromatin modifier, an ATP-dependent chromatin remodeling complex, a histone kinase, a histone phosphorylase, a histone ubiquitin ligase, and a histone small ubiquitin-like modifier (SUMO) modifying enzyme. In certain embodiments that may be combined with any of the preceding embodiments, the recombinant polypeptide complexes with at least two H3K4 histone demethylases. In certain embodiments, the at least two H3K4 histone demethylases are LDL1 and LDL2.

In certain embodiments that may be combined with any of the preceding embodiments, the recombinant polypeptide has methyltransferase activity. In certain embodiments, the recombinant polypeptide methylates H3K9. In certain embodiments that may be combined with any of the preceding embodiments, the DNA-binding activity of the recombinant polypeptide is modified by one or more hormones or external stimuli. In certain embodiments, the one or more hormones are selected from auxin, ethylene, gibberellin, jasmonic acid, brassinosteroid, and ABA. In certain embodiments, the one or more external stimuli are selected from, plant dehydration, plant wounding, cold temperatures, and fungi. In certain embodiments that may be combined with any of the preceding embodiments, the DNA-binding activity of the recombinant polypeptide is induced by the one or more hormones or external stimuli. In certain embodiments that may be combined with any of the preceding embodiments, the DNA-binding activity of the recombinant polypeptide is repressed by the one or more hormones or external stimuli.

In certain embodiments that may be combined with any of the preceding embodiments, the one or more target nucleic acids are endogenous nucleic acids. In certain embodiments that may be combined with any of the preceding embodiments, the one or more target nucleic acids are transgenes. In certain embodiments that may be combined with any of the preceding embodiments, expression of the one or more target nucleic acids is silenced.

Other aspects of the present disclosure relate to a recombinant nucleic acid encoding an SUVR5-like protein, where the SUVR5-like protein contains a DNA-binding domain, a C-terminal pre-SET domain, a C-terminal SET domain, and a C-terminal post-SET domain; and where the DNA-binding domain is heterologous to an SUVR5 DNA-binding domain.

Other aspects of the present disclosure relate to a recombinant nucleic acid encoding an SUVR5-like protein containing a DNA-binding domain, a C-terminal pre-SET domain, a C-terminal SET domain, and a C-terminal post-SET domain; and where the DNA-binding domain is not SEQ ID NO: 1.

Other aspects of the present disclosure relate to a recombinant nucleic acid encoding an SUVR5-like protein containing a DNA-binding domain, a C-terminal pre-SET domain, a C-terminal SET domain, and a C-terminal post-SET domain; and where the DNA-binding domain is any DNA-binding domain other than SEQ ID NO: 1.

Other aspects of the present disclosure relate to a recombinant nucleic acid encoding an SUVR5-like protein containing a DNA-binding domain, a C-terminal pre-SET domain, a C-terminal SET domain, and a C-terminal post-SET domain; and where the DNA-binding domain contains an amino acid sequence that is less than 99%, less than 98%, less than 97%, less than 96%, less than 95%, less than 94%, less than 93%, less than 92%, less than 91%, less than 90%, less than 85%, less than 80%, or less than 75% identical to SEQ ID NO: 1.

Other aspects of the present disclosure relate to a recombinant nucleic acid encoding an SUVR5-like protein containing a DNA-binding domain, a C-terminal pre-SET domain, a C-terminal SET domain, and a C-terminal post-SET domain; and where the DNA-binding domain contains an amino acid sequence with at least one, at least two, at least three, at least four, or at least five amino acid differences as compared to the amino acid sequence of SEQ ID NO: 1.

Other aspects of the present disclosure relate to a recombinant nucleic acid encoding an SUVR5-like protein, where the SUVR5-like protein contains a DNA-binding domain, a C-terminal pre-SET domain, a C-terminal SET domain, and a C-terminal post-SET domain; and where the DNA-binding domain binds with a sequence specificity other than that of SEQ ID NO: 1.

In certain embodiments that may be combined with any of the preceding embodiments, the DNA-binding domain contains a zinc finger domain. In certain embodiments, the zinc finger domain contains two, three, four, five, six, seven, eight, or nine zinc fingers. In certain embodiments, the zinc finger domain is a zinc finger array. In certain embodiments, the zinc finger domain is selected from a C2H2 zinc finger domain, a CCCH zinc finger domain, a multi-cysteine zinc finger domain, and a zinc binuclear cluster domain. In certain embodiments that may be combined with any of the preceding embodiments, the DNA-binding domain is selected from a TAL effector targeting domain, a helix-turn-helix family DNA-binding domain, a basic domain, a ribbon-helix-helix domain, a TBP domain, a barrel dimer domain, a real homology domain, a BAH domain, a SANT domain, a Chromodomain, a Tudor domain, a Bromodomain, a PHD domain, a WD40 domain, and a MBD domain. In certain embodiments that may be combined with any of the preceding embodiments, the DNA-binding domain contains a TAL effector targeting domain.

In certain embodiments that may be combined with any of the preceding embodiments, the SUVR5-like protein further contains one or more additional DNA-binding domains. In certain embodiments, the one or more additional DNA-binding domains contain an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1. In certain embodiments, the one or more additional DNA-binding domains contain an amino acid sequence 100% identical to SEQ ID NO: 1. In certain embodiments, the one or more additional DNA-binding domains contain an amino acid sequence heterologous to SEQ ID NO: 1.

In certain embodiments that may be combined with any of the preceding embodiments, the C-terminal pre-SET domain contains an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 2 or SEQ ID NO: 3. In certain embodiments that may be combined with any of the preceding embodiments, the C-terminal SET domain contains an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to an amino acid sequence selected from SEQ ID NOs: 4, 5, and 6. In certain embodiments that may be combined with any of the preceding embodiments, the C-terminal post-SET domain contains an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 7.

In certain embodiments that may be combined with any of the preceding embodiments, the modified SUVR5-like protein complexes with one or more polypeptides selected from a DNA methyltransferase, a histone methyltransferase, a histone deacetylase, a histone demethylase, a chromatin modifier, an ATP-dependent chromatin remodeling complex, a histone kinase, a histone phosphorylase, a histone ubiquitin ligase, and a histone small ubiquitin-like modifier (SUMO) modifying enzyme. In certain embodiments that may be combined with any of the preceding embodiments, the modified SUVR5-like protein complexes with at least two H3K4 histone demethylases. In certain embodiments that may be combined with any of the preceding embodiments, the at least two H3K4 histone demethylases are LDL1 and LDL2.

In certain embodiments that may be combined with any of the preceding embodiments, the modified SUVR5-like protein has methyltransferase activity. In certain embodiments, the modified SUVR5-like protein methylates H3K9. In certain embodiments that may be combined with any of the preceding embodiments, the DNA-binding activity of the modified SUVR5-like protein is modified by one or more hormones or external stimuli. In certain embodiments, the one or more hormones are selected from auxin, ethylene, gibberellin, jasmonic acid, brassinosteroid, and ABA. In certain embodiments, the one or more external stimuli are selected from, plant dehydration, plant wounding, cold temperatures, and fungi. In certain embodiments that may be combined with any of the preceding embodiments, the DNA-binding activity of the recombinant polypeptide is induced by the one or more hormones or external stimuli. In certain embodiments that may be combined with any of the preceding embodiments, the DNA-binding activity of the recombinant polypeptide is repressed by the one or more hormones or external stimuli.

In certain embodiments that may be combined with any of the preceding embodiments, the DNA-binding domain binds one or more target nucleic acids. In certain embodiments, the one or more target nucleic acids are polypeptide-encoding nucleic acids. In certain embodiments, the one or more target nucleic acids are endogenous plant nucleic acids. In certain embodiments, the one or more target nucleic acids are plant transgenes. In certain embodiments that may be combined with any of the preceding embodiments, the modified SUVR5-like protein reduces expression of the one or more target nucleic acids. In certain embodiments that may be combined with any of the preceding embodiments, the modified SUVR5-like protein silences expression of the one or more target nucleic acids.

Other aspects of the present disclosure relate to a vector containing the recombinant nucleic acid of any of the preceding embodiments, where the recombinant nucleic acid is operably linked to a regulatory sequence. Other aspects of the present disclosure relate to a host cell containing the expression vector of the proceeding embodiment. In certain embodiments, the host cell is a plant cell. Other aspects of the present disclosure relate to a recombinant plant containing the recombinant nucleic acid of any of the proceeding embodiments.

### BRIEF DESCRIPTION OF THE DRAWINGS

FIG. 1 depicts the overexpression of FLC and the late flowering phenotype of suvr5 mutants. FIG. 1A depicts a picture showing the late flowering phenotype of suvr5-1 mutants. FIG. 1B depicts an analysis of the late flowering phenotype of suvr5-1 mutants by scoring the number of leaves at bolting and standard error (SE). FIG. 1C depicts results from RT-qPCR showing FLC expression levels relative to ACTIN in 3-week-old Col0 and suvr5-1 mutant plant leaves (triplicate mean and SE are shown).

FIG. 2 depicts a SAM binding assay showing that the SUVR5 SET domain binds the methyl group donor S-adenosyl-1-[methyl-³H]methionine and that this interaction is lost upon mutation of amino acid residue 1307 from His to Leu.

FIG. 3 depicts a ClustalW alignment of A. thaliana SUVR5 and its homologs, showing that SUVR5 is conserved in plant species, including moss, but not algae.

FIG. 4 schematically depicts the SELEX experimental procedure.

FIG. 5 depicts the sequencing results obtained from the SELEX experiment.

FIG. 6 schematically depicts the genomic-SELEX experimental procedure.

FIG. 7A schematically depicts the domain structure of the A. thaliana SUVR5. FIG. 7B depicts the enriched motifs identified in sequencing data obtained from SELEX experiments. FIG. 7C depicts meta-gene analysis of genomic-SELEX reads showing preferential binding of the SUVR5 zinc finger domain to the region upstream protein coding genes (PCG). The results obtained after exponential selection of the binding sites for 9 cycles are shown (x9) in contrast with the results obtained after only one cycle of enrichment (xi), included as control of the initial DNA population used for the experiment. FIG. 7D left depicts mobility shift assays with increasing amounts of GST-zinc finger domain (100, 250, and 500 ng) added to a binding reaction with either an unspecific oligonucleotide probe or a probe including the identified binding motif sequence. FIG. 7D right depicts binding of SUVR5 zinc fingers to the specific probe and point mutations of the specific probe.

FIG. 8 shows the partial redundancy of SUVR5 function with HMTases SUVH4, SUVH5, and SUVH6. FIG. 8A depicts chromosomal views of the log 2 ratio of suvr5 mutants to WT in red, and chromosomal views of the log 2 ratio of the suvh4 suvh5 suvh6 triple mutants to WT in black. FIG. 8B depicts meta-analysis of H3K9me2 levels on suvh456 and suvr5 mutants vs. WT over TEs. FIG. 8C depicts the developmental defects caused by mutation of the four SET domain proteins SUVH4, SUVH5, SUVH6, and SUVR5 (even if only heterozygous for one of them). FIG. 8D depicts a genome browser view of a region in the arms of chromosome 1. H3K9me2 data is represented as log 2 ratios from 0 to 3. Gene models correspond to TAIR8 protein-coding genes (PCG) and are shown for the plus or minus strand of the genome

FIG. 9 shows that SUVR5 specific H3K9me2 deposition correlates with zinc finger domain binding and promotes gene silencing. FIG. 9A depicts a Venn diagram representation of the number of H3K9me2 decreased regions found in suvr5 mutants that are specific or overlap with the ones in the suvh4 suvh5 suvh6 triple mutant. FIG. 9B depicts a genome browser view of the region around AT3G22121. H3K9me2 data is represented as log 2 ratios from 0 to 2.5. Gene models correspond to TAIR8 protein-coding genes (PCG). FIG. 9C depicts box plots showing the levels of H3K9me2 in the genes that have genomic SELEX signal in their upstream 3 Kb regions (left panel, results for the ChIP-chip first replicate; right panel, results for the second replicate. In both cases the decrease is significant with a P<0.01). FIG. 9D depicts box plots showing the expression levels (in RPKM) of genes in Col0 and suvr5-1 mutants. Left panel, results for all genes; right panel, results for the 444 genes that overlap with the defined H3K9me2 regions.

FIG. 10 shows examples of genes that show decreased H3K9me2 levels and increased expression. Depicted are results from ChIP-chip experiments, ChIP-chip validation by single locus ChIP qPCR, and ChIP-chip validation by mRNAseq RT-qPCR.

FIG. 11 shows that SUVR5 H3K9me2 deposition is independent of DNA methylation. FIG. 11A depicts the chromosome-wide distribution of methylation in suvr5-1 vs. Col0. FIG. 11B depicts meta analysis of CG, CHG, and CHH DNA methylation levels in the defined suvr5-specific H3K9me2 decreased regions and their upstream and downstream areas.

FIG. 12 depicts the characterization of the mutant alleles suvr5-1 (see, Joshua S. Mylne, Lynne Barrett, Federico Tessadori, Stéphane Mesnage, Lianna Johnson, Yana V. Bernatavichute, Steven E. Jacobsen, Paul Fransz and Caroline Dean, (2006) LHP1, the Arabidopsis homologue of HETEROCHROMATIN PROTEIN1, is required for epigenetic silencing of FLC. Proc. Nat. Acad. Sci. U.S.A. 103: 5012-5017) and suvr5-2.

FIG. 13 shows that SUVR5 significantly affects genes related to the “response to stimulus” GO term cluster. FIG. 13A depicts an AgriGO GO flash chart showing the biological process GO term clustering of the genes upregulated in suvr5 (suvr5 vs. Col0 over 4 fold, P<0.01). The highlighted categories correspond to the significant ones (based on FDR). FIG. 13B depicts a picture of Col0, suvr5-1 and suvr5-2 seedlings after 0.5 μM NAA treatment. FIG. 13C depicts a time course root length measurements of Col0, suvr5-1 and suvr5-2 seedlings before and after NAA addition. The bottom right panel shows the slopes of the curves that represent a measurement of the growth rate. Around 20 seedlings of each line were measured and SE are shown for every point. FIG. 13D depicts the expression levels of 3 selected auxin-responsive genes in seedlings grown for 13 days without NAA application (CONTROL) or transferred to NAA media on the sixth day (+NAA 0.5 μM).

FIG. 14 depicts a chart showing the GO term categories included in the “response to stimulus” cluster. The p-values showing the level of significance of the over-representation of that GO term in the set of suvr5 vs. Col0 upregulated genes compared to the whole genome are shown in parentheses. At the bottom of each box, the number of genes that include the particular GO term in the suvr5 upregulated set of genes/total number of suvr5 upregulated genes is shown on the left; and the number of genes that include the particular GO term in the whole genome/total number of genes in the whole genome (i.e., color intensity increases with significance) is shown on the right.

FIG. 15 shows that SUVR5 and LDL1 act together in a repressor complex. FIG. 15A depicts analysis of the late flowering phenotype of ldl1 ldl2 mutants and its complementation by the tagged LDL1 transgene by scoring number of leaves at bolting. FIG. 15B depicts a table showing the mass spectrometry analysis of LDL1 affinity purifications. FIG. 15C depicts a picture showing the late flowering phenotype of suvr5 mutant plants, ldl1 ldl2 double mutant plants, and suvr5 ldl1 ldl2 triple mutant plants. FIG. 15D depicts analysis of the late flowering phenotype by scoring number of leaves at bolting. FIG. 15E depicts a box plot showing the expression level (in RPKM) of the 270 genes upregulated in the suvr5 mutant and ldl1 ldl2 double mutant (over 4 fold and P<0.01 for both, suvr5/Col0 and ldl1 ldl2/Col0) in Col0, suvr5, the ldl1 ldl2 double, and the suvr5 ldl1 ldl2 triple mutants, showing the epistatic and not synergistic relationship between the mutants.

FIG. 16 depicts an AgriGO GO flash chart showing the biological process GO term clustering of the genes upregulated in the ldl1 ldl2 double mutant (ldl1 ldl2 vs. Col0 over 4 fold, P<0.01). The highlighted categories correspond to significant ones based on FDR.

FIG. 17 schematically depicts the model for SUVR5 function.

FIG. 18A schematically depicts the relationship between DNA methylation and H3K9me2 of the FWA promoter repeats and flowering time in wild type Col0 plants and fwa-4 epimutant plants. In fwa-4 mutant plants, a loss of DNA and histone methylation at the promoter leads to activation of FWA gene expression, which delays flowering time. FIG. 18B schematically depicts the construct generated to express a form of SUVR5 where its own zinc fingers (amino acids 730 to 860) have been replaced by the 108 zinc finger (ZF) that targets the protein to the repeats in the FWA promoter. FIG. 18C depicts the partial reversal of the late flowering phenotype of fwa-4 mutants when the ZF-SUVR5 protein is transformed into fwa-4 plants.

### DETAILED DESCRIPTION

### Overview

The present disclosure relates to methods for reducing expression of one or more target nucleic acids in a plant, by providing a plant containing a recombinant polypeptide, where the recombinant polypeptide contains a DNA-binding domain, a C-terminal pre-SET domain, a C-terminal SET domain, and a C-terminal post-SET domain; and growing the plant under conditions whereby the recombinant polypeptide binds to the one or more target nucleic acids, thereby reducing expression of the one or more target nucleic acids. The present disclosure also relates to a recombinant nucleic acid encoding an SUVR5-like protein, where the SUVR5-like protein contains a DNA-binding domain, a C-terminal pre-SET domain, a C-terminal SET domain, and a C-terminal post-SET domain, and where the DNA-binding domain is heterologous to an SUVR5 DNA-binding domain; the DNA-binding domain is not the endogenous DNA-binding domain of SUVR5 (i.e., is not SEQ ID NO: 1); the DNA-binding domain is any DNA-binding domain other than SEQ ID NO: 1; the DNA-binding domain contains an amino acid sequence that is less than 99%, less than 98%, less than 97%, less than 96%, less than 95%, less than 94%, less than 93%, less than 92%, less than 91%, less than 90%, less than 85%, less than 80%, or less than 75% identical to SEQ ID NO: 1; the DNA-binding domain contains an amino acid sequence with at least one, at least two, at least three, at least four, or at least five amino acid differences as compared to the amino acid sequence of SEQ ID NO: 1; or the DNA-binding domain binds with a sequence specificity other than that of SEQ ID NO: 1. The present disclosure further relates to vectors containing such recombinant nucleic acids, host cells containing such recombinant nucleic acids and vectors, and recombinant plants containing such recombinant nucleic acids.

Moreover, the present disclosure is based, at least in part, on the novel discovery of an Arabidopsis thaliana methyltransferase (SUVR5) that functions by recognizing a specific DNA sequence through a domain that includes three zinc fingers in tandem. Additionally, it was shown that SUVR5 is responsible for changes in methylation of histone H3 lysine 9 (H3K9). It is believed that SUVR5 is a natural recruiter of silencing complexes that tethers them to sequence-specific locations throughout the genome. Advantageously, SUVR5 activity can be modulated by plant hormones and environmental stimuli. Moreover, a modified SUVR5 can be engineered to specifically bind different DNA sequences by replacing the endogenous DNA-binding zinc finger domain with a heterologous DNA-binding domain, such as heterologous zinc finger domains or TAL effector targeting domains. Alternatively, a gene of interest may be engineered to be operably linked to a control region, such as a promoter, that contains the SUVR5-binding sequence.

### Definitions

Unless defined otherwise, all scientific and technical terms are understood to have the same meaning as commonly used in the art to which they pertain. For the purpose of the present disclosure, the following terms are defined.

As used herein, an “SUVR5-like protein” refers to a recombinant protein that has similar activity to an SUVR5 protein, such as the A. thaliana SUVR5 protein, but contains a DNA-binding domain that is heterologous to a naturally-occurring (i.e., endogenous) SUVR5 DNA-binding domain.

As used herein, a “target nucleic acid” refers to a portion of double-stranded polynucleotide acid, e.g., RNA, DNA, PNA (peptide nucleic acid) or combinations thereof, to which it is advantageous to bind a protein, such as an SUVR5 protein. In one embodiment, a “target nucleic acid” is all or part of a transcriptional control element for a gene for which a desired phenotypic result can be attained by altering the degree of its expression. A transcriptional control element includes positive and negative control elements such as a promoter, an enhancer, other response elements, e.g., steroid response element, heat shock response element, metal response element, a repressor binding site, operator, and/or a silencer. The transcriptional control element can be viral, eukaryotic, or prokaryotic. A “target nucleic acid” also includes a downstream nucleic acid that can bind a protein and whose expression is thereby modulated, typically preventing transcription.

As used herein, a “target gene” refers to a gene whose expression is to be reduced by an SUVR5 protein or an SUVR5-like protein in plant cells.

As used herein, the terms “polynucleotide”, “nucleic acid”, “nucleic acid sequence”, “sequence of nucleic acids”, and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; inter-nucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

As used herein, a “polypeptide” is an amino acid sequence containing a plurality of consecutive polymerized amino acid residues (e.g., optionally at least about 15 consecutive polymerized amino acid residues, at least about 30 consecutive polymerized amino acid residues, or at least about 50 consecutive polymerized amino acid residues). In many instances, a polypeptide contains a polymerized amino acid residue sequence that is an enzyme, a methyltransferase, a demethylase, a deacteylase, a predicted protein of unknown function, or a domain or portion or fragment thereof. The polypeptide optionally contains modified amino acid residues, naturally occurring amino acid residues not encoded by a codon, and non-naturally occurring amino acid residues.

As used herein, “protein” refers to an amino acid sequence, oligopeptide, peptide, polypeptide, or portions thereof whether naturally occurring or synthetic.

Genes and proteins that may be used in the present disclosure include genes encoding conservatively modified variants and proteins that are conservatively modified variants of those genes and proteins described throughout the application. “Conservatively modified variants” as used herein include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Homologs of the genes and proteins described herein may also be used in the present disclosure. As used herein, “homology” refers to sequence similarity between a reference sequence and at least a fragment of a second sequence. Homologs may be identified by any method known in the art, preferably, by using the BLAST tool to compare a reference sequence to a single second sequence or fragment of a sequence or to a database of sequences. As described below, BLAST will compare sequences based upon percent identity and similarity. As used herein, “orthology” refers to genes in different species that derive from a common ancestor gene.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 29% identity, optionally 30%, 40%, 45%, 50%, 55%, 60%, 65%, 70%, 75%, 80%, 85%, 90%, 95%, 99% or 100% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200, or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For blastn, the default parameters are Gap opening penalty=5 and Gap extension penalty=2. For blastp, the default parameters are Gap opening penalty=11 and Gap extension penalty=1.

A “comparison window,” as used herein, includes reference to a segment of any one of the number of contiguous positions including, but not limited to from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1981), by the homology alignment algorithm of Needleman and Wunsch (1970) J Mol Biol 48(3):443-453, by the search for similarity method of Pearson and Lipman (1988) Proc Natl Acad Sci USA 85(8):2444-2448, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection [see, e.g., Brent et al., (2003) Current Protocols in Molecular Biology, John Wiley & Sons, Inc. (Ringbou Ed)].

Two examples of algorithms that are suitable for determining percent sequence identity and sequence similarity are the BLAST and BLAST 2.0 algorithms, which are described in Altschul et al. (1997) Nucleic Acids Res 25(17):3389-3402 and Altschul et al. (1990) J. Mol Biol 215(3)-403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=-4, and a comparison of both strands. For amino acid sequences, the BLASTP program uses as defaults a wordlength of 3, and expectation (E) of 10, and the BLOSUM62 scoring matrix (see Henikoff and Henikoff, (1992) Proc Natl Acad Sci USA 89(22):10915-10919) alignments (B) of 50, expectation (E) of 10, M=5, N=−4, and a comparison of both strands.

The BLAST algorithm also performs a statistical analysis of the similarity between two sequences (see, e.g., Karlin and Altschul, (1993) Proc Natl Acad Sci USA 90(12):5873-5877). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Other than percentage of sequence identity noted above, another indication that two nucleic acid sequences or polypeptides are substantially identical is that the polypeptide encoded by the first nucleic acid is immunologically cross-reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

### SUVR5 Proteins of the Present Disclosure

Certain aspects of the present disclosure relate to recombinant SUVR5 proteins that contain a DNA-binding domain, a C-terminal pre-SET domain, a C-terminal SET domain, and a C-terminal post-SET domain; and to the use of such proteins in reducing the expression of one or more target nucleic acids, such as genes, in plants.

SUVR5 proteins of the present disclosure are members of the Su(var)3-9 family of methyltransferases and methyltransferase homologs. As used herein, a “methyltransferase” is an enzyme that catalyzes the transfer of a methyl group from a donor, such as S-adenosyl methionine, to an acceptor, such as a nucleic acid or protein. Methyltransferases include, without limitation, DNA methyltransferases and histone methyltransferases. In certain embodiments, SUVR5 proteins of the present disclosure have methyltransferase activity. In certain preferred embodiments, SUVR5 proteins of the present disclosure methylate histone H3 lysine 9 (H3K9).

In other embodiments, an SUVR5 protein of the present disclosure is a functional fragment that maintains the binding specificity and catalytic activity of the corresponding full length SUVR5 protein.

Suitable SUVR5 proteins may be identified and isolated from monocot and dicot plants. Examples of such plants include, without limitation, Arabidopsis spp., Ricinus communis, Glycine max, Zea Mays, Medicago truncatula, Physcomitrella patens, Sorghum bicolor, and Oryza sativa. Examples of suitable SUVR5 proteins include, without limitation, those listed in Table 1, homologs thereof, and orthologs thereof.

TABLE 1
SUVR5 Proteins

| | | Polypeptide |
|---|---|---|
| Organism | Gene Name | SEQ ID NO: |
| Ricinus communis | 29676.t000093 | 9 |
| Glycine max | Glyma11g06620 | 10 |
| Glycine max | Glyma02g06760 | 11 |
| Glycine max | Glyma16g25800 | 12 |
| Glycine max | Glyma01g38670 | 13 |
| Zea mays | GRMZM2G172427 | 14 |
| Zea mays | GRMZM2G125432 | 15 |
| Medicago truncatula | Medtr8g147270 | 16 |
| Medicago truncatula | Medtr5g018800 | 17 |
| Medicago truncatula | Medtr8g094130 | 18 |
| Medicago truncatula | AC233653_3 | 19 |
| Medicago truncatula | AC233653_10 | 20 |
| Physcomitrella patens | Pp1s174_93V6 | 21 |
| Physcomitrella patens | Pp1s100_44V6 | 22 |
| Physcomitrella patens | Pp1s325_74V6 | 23 |
| Sorghum bicolor | Sb04g030350 | 24 |
| Oryza sativa ssp. Japonica | LOC_Os02g47900 | 25 |
| Setaria italica | Si016095m | 26 |
| Brachypodium distachyon | Bradi3g52950 | 27 |
| Manihot esculenta | cassava4.1_000198m.g | 28 |
| Populus trichocarpa | POPTR_0005s13810 | 29 |
| Citrus sinensi | orange1.1g000416m.g | 30 |
| Citrus clementina | clementine0.9_000274m.g | 31 |
| Vitis vinifera | GSVIVG01019046001 | 32 |
| Prunus persica | ppa000179m.g | 33 |
| Mimulus guttatus | mgv1a000212m.g | 34 |
| Cucumis sativus | Cucsa.101850 | 35 |
| Carica papaya | evm.TU.supercontig_15.107 | 36 |
| Eucalyptus grandis | Eucgr.H01928 | 37 |
| Arabidopsis lyrata | 481235 | 38 |

In certain embodiments, the SUVR5 protein is the Arabidopsis thaliana SUVR5 protein, which is a 155 kDa protein that contains a DNA-binding domain having three C2H2 zinc fingers in tandem, a C-terminal pre-SET domain, a C-terminal SET domain, and a C-terminal post-SET domain.

In other embodiments, an SUVR protein of the present disclosure has an amino acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of the A. thaliana SUVR5 protein (i.e., SEQ ID NO: 8).

### DNA-Binding Domains

SUVR5 proteins of the present disclosure have DNA-binding activity. This DNA-binding activity is achieved through a DNA-binding domain. In certain embodiments, SUVR5 proteins of the present disclosure contain a DNA-binding domain. SUVR5 proteins of the present disclosure may contain one DNA binding domain or they may contain more than one DNA-binding domain.

SUVR5 proteins of the present disclosure contain a DNA-binding domain. In certain embodiments, the DNA-binding domain is the endogenous domain that occurs naturally in SUVR5 proteins of the present disclosure. In other embodiments, the SUVR5 protein is a modified protein that contains a heterologous (i.e., is non-naturally occurring or is not endogenous in a SUVR5 protein) DNA-binding domain.

In certain embodiments, the DNA-binding domain is a zinc finger domain. As disclosed herein, a “zinc finger domain” refers to a DNA-binding protein domain that contains zinc fingers, which are small protein structural motifs that can coordinate one or more zinc ions to help stabilize their protein folding. Zinc fingers can generally be classified into several different structural families and typically function as interaction modules that bind DNA, RNA, proteins, or small molecules. Suitable zinc finger domains of the present disclosure may contain two, three, four, five, six, seven, eight, or nine zinc fingers. Examples of suitable zinc finger domains include, without limitation, Cys2His2 (C2H2) zinc finger domains, C-x8-C-x5-C-x3-H (CCCH) zinc finger domains, multi-cysteine zinc finger domains, and zinc binuclear cluster domains.

In certain embodiments, the SUVR5 protein contains a zinc finger domain having three C2H2 fingers. In some embodiments, the zinc finger domain having three C2H2 fingers has an amino acid sequence that is at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to the amino acid sequence of the DNA-binding domain of A. thaliana SUVR5 (i.e., SEQ ID NO: 1). In other embodiments, the first C2H2 finger has an amino acid sequence that is at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to amino acids 11-33 of SEQ ID NO: 1 or to amino acids 9-32 of SEQ ID NO: 1. In yet other embodiments, the second C2H2 finger has an amino acid sequence that is at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to amino acids 45-66 of SEQ ID NO: 1 or to amino acids 43-66 of SEQ ID NO: 1. In further embodiments, the third C2H2 finger has an amino acid sequence that is at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to amino acids 114-134 of SEQ ID NO: 1 or to amino acids 112-134 of SEQ ID NO: 1.

In other embodiments, the DNA-binding domain binds a specific nucleic acid sequence. For example, the DNA-binding domain may bind a sequence that is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 20 nucleotides, at least 25 nucleotides, at least 30 nucleotides, at least 35 nucleotides, at least 40 nucleotides, at least 45 nucleotides, at least 50 nucleotides, or a high number of nucleotides in length. In certain embodiments, the DNA-binding domain binds a sequence that is 8 nucleotides in length. In certain preferred embodiments, the DNA-binding domain binds the nucleic acid sequence: TACTAGTA.

In other embodiments, a SUVR5 protein of the present disclosure further contains two N-terminal CCCH zinc finger domains.

In some embodiments where the SUVR5 protein contains a heterologous DNA-binding domain, the zinc finger domain is an engineered zinc finger array, such as a C2H2 zinc finger array. Engineered arrays of C2H2 zinc fingers can be used to create DNA-binding proteins capable of targeting desired genomic DNA sequences. Methods of engineering zinc finger arrays are well known in the art, and include, for example, combining smaller zinc fingers of known specificity.

In other embodiments where the SUVR5 protein contains a heterologous DNA-binding domain, the SUVR5 protein may contain a DNA-binding domain other than a zinc finger domain. Examples of such DNA-binding domains include, without limitation, TAL (transcription activator-like) effector targeting domains, helix-turn-helix family DNA-binding domains, basic domains, ribbon-helix-helix domains, TBP (TATA-box binding protein) domains, barrel dimer domains, RHB domains (real homology domain), BAH (bromo-adjacent homology) domains, SANT domains, Chromodomains, Tudor domains, Bromodomains, PHD domains (plant homeo domain), WD40 domains, and MBD domains (methyl-CpG-binding domain).

In certain preferred embodiments where the SUVR5 protein contains a heterologous DNA-binding domain, the DNA-binding is a TAL effector targeting domain. As used herein, TAL effectors refer to secreted bacterial proteins, such as those secreted by Xanthomonas or Ralstonia bacteria when infecting various plant species. Generally, TAL effectors are capable of binding promoter sequences in the host plant, and activate the expression of plant genes that aid in bacterial infection. TAL effectors recognize plant DNA sequences through a central repeat targeting domain that contains a variable number of approximately 34 amino acid repeats. Moreover, TAL effector targeting domains can be engineered to target specific DNA sequences. Methods of modifying TAL effector targeting domains are well known in the art, and described in Bogdanove and Voytas, Science. 2011 Sep. 30; 333(6051):1843-6.

SUVR5 proteins of the present disclosure may contain more than one DNA-binding domain. In certain embodiments, at least one of the DNA-binding domains is the endogenous DNA-binding domain that occurs naturally in a SUVR5 protein. In certain embodiments, at least one of the DNA-binding domains is a heterologous (i.e., is non-naturally occurring or is not endogenous in a SUVR5 protein) DNA-binding domain. In certain preferred embodiments, SUVR5 proteins of the present disclosure contain one additional DNA-binding domain in addition to the endogenous DNA-binding domain. In certain preferred embodiments, SUVR5 proteins of the present disclosure with more than one DNA-binding domain contain both an endogenous DNA-binding domain and a heterologous DNA-binding domain.

SUVR5 proteins of the present disclosure that contain more than one DNA-binding domain may contain, for example, one or more, two or more, three or more, four or more, or five or more additional DNA-binding domains. It is to be understood that the one or more additional DNA-binding domains in SUVR5 proteins may have similar or identical characteristics and/or properties as described for a single DNA-binding domain in a SUVR5 protein. The one or more additional DNA-binding domains may include, for example, any of the zinc finger domains disclosed herein, any of the TAL effector targeting domains disclosed herein, any of the helix-turn-helix family DNA-binding domains disclosed herein, any of the basic domains disclosed herein, any of the ribbon-helix-helix domains disclosed herein, any of the TBP domains disclosed herein, any of the barrel dimer domains disclosed herein, any of the real homology domains disclosed herein, any of the BAH domains disclosed herein, any of the SANT domains disclosed herein, any of the Chromodomains disclosed herein, any of the Tudor domains disclosed herein, any of the Bromodomains disclosed herein, any of the PHD domains disclosed herein, any of the WD40 domains disclosed herein, and/or any of the MBD domains disclosed herein. The one or more additional DNA-binding domains may, for example, bind a particular nucleic acid sequence as described for a DNA-binding domain.

SUVR5 DNA-Binding Activity

Other aspects of the present disclosure relate to SUVR5 proteins whose DNA-binding activity in plants can be modified by plant hormones or external stimuli. Without wishing to be bound by theory, it is believed that plant hormones and external stimuli modify the activity of SUVR5 proteins by inducing/repressing or upregulating/downregulating plant hormone-induced or external stimuli-induced genes that affect SUVR5 protein activity by, for example, protein degradation, activating/inactivating post-translational modifications, or increasing/decreasing the DNA-binding ability of the SUVR5 protein. In certain embodiments, the hormones or external stimuli induce SUVR5 DNA-binding activity. In other embodiments, the hormones or external stimuli repress SUVR5 DNA-binding activity. Without wishing to be bound by theory, it is believed that the type of hormone or external stimuli that is used determines whether the DNA-binding activity is induced or repressed.

Examples of plant hormones that are capable of modifying SUVR5 DNA-binding activity include, without limitation, auxin, ethylene, gibberellin, jasmonic acid, brassinosteroid, and ABA (abscisic acid). Examples of external stimuli that are capable of modifying SUVR5 DNA-binding activity include, without limitation, plant dehydration, plant wounding, cold temperatures, and fungi.

SET Domains

SUVR5 proteins of the present disclosure also contain a C-terminal pre-SET domain, a C-terminal SET domain, and a C-terminal post-SET domain. As disclosed herein, a SET (Su(var)3-9, Enhancer-of-zeste, Trithorax) domain is a protein domain that has lysine methyltransferase activity.

SET domains of the present disclosure may contain a series of β-strands folding into three discrete sheets that surround a knot-like structure (e.g., Taylor, W. R. et al. Comput. Biol. Chem. 27, 11-15, 2003). Generally, the knot-like structure is formed by the C-terminal segment of the SET domain passing through a loop formed by a preceding stretch of the sequence. The C-terminal segment and the loop contain the two most conserved sequence motifs in the SET domains. The conserved motifs are: ELxF/YDY and NHS/CxxPN, where “x” is any amino acid) (e.g., C. Qian and M.-M. Zhou. Cell Mol Life Sci 63:2755-2763, 2006).

In certain embodiments, a SET domain of the present disclosure has an amino acid sequence that is at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to SEQ ID NO: 4. In other embodiments, a SET domain of the present disclosure has an amino acid sequence that is at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to SEQ ID NO: 5. In further embodiments, a SET domain of the present disclosure has an amino acid sequence that is at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to SEQ ID NO: 6.

SET domains of the present disclosure are generally flanked by a pre-SET domain and a post-SET domain. As used herein, a pre-SET domain is a cysteine-rich zinc-binding domain that occurs N-terminal to a SET domain.

Pre-SET domains of the present disclosure, such as those found in the SUV39 SET family, may contain nine invariant cysteine residues that are grouped into two segments separated by a region of variable length. These 9 cysteines coordinate 3 zinc ions to form a triangular cluster, where each of the zinc ions is coordinated by 4 four cysteines to give a tetrahedral configuration. The function of this domain is structural, holding together 2 long segments of random coils and stabilizing the SET domain.

In certain embodiments, a pre-SET domain of the present disclosure has an amino acid sequence that is at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to SEQ ID NO: 2. In other embodiments, a pre-SET domain of the present disclosure has an amino acid sequence that is at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to SEQ ID NO: 3.

As used herein, a post-SET domain is a cysteine-rich zinc-binding domain that occurs following (i.e., C-terminal to) a SET domain.

Generally, post-SET domains of the present disclosure are disordered when not interacting with a histone tail and in the absence of zinc. Post-SET domains of the present disclosure may contain three conserved cysteines that form a zinc-binding site when coupled to a fourth conserved cysteine in the knot-like structure close to the SET domain active site. The structured post-SET region brings in the C-terminal residues that participate in S-adenosylmethine-binding and histone tail interactions. The three conserved cysteine residues are essential for HMTase activity, as it has been previously shown that replacement with serine abolishes HMTase activity.

In certain embodiments, a post-SET domain of the present disclosure has an amino acid sequence that is at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to SEQ ID NO: 7.

SUVR5 Protein Complexes

Further aspects of the present disclosure relate to SUVR5 proteins that are capable of complexing with one or more proteins. Without wishing to be bound by theory, it is believed that in plants SUVR5 proteins of the present disclosure recruit gene silencing protein complexes and tether the complexes to specific DNA sequences.

Accordingly, in certain embodiments, an SUVR5 protein of the present disclosure complexes with one or more, two or more, three or more, four or more, five or more, six or more, seven or more, eight or more, nine or more, or ten or more proteins. Examples of suitable proteins that can be in complex with an SUVR5 protein of the present disclosure includes, without limitation, DNA methyltransferases, histone methyltransferases, histone deacetylases, histone demethylases, other chromatin modifiers, ATP-dependent chromatin remodeling complexes, histone kinases, histone phosphorylases, histone ubiquitin ligases, and histone SUMO (small ubiquitin-like modifier) modifying enzyme.

In certain embodiments, an SUVR5 protein of the present disclosure complexes with at least one, at least two, at least three, at least four, at least five, or more H3K4 histone demethylases. In some embodiments, the SUVR5 protein complexes with the lysine-specific H3K4 histone demethylase LDL1 (e.g., see Spedaletti V, et al. Biochemistry, 2008). In other embodiments, the SUVR5 protein complexes with the H3K4 histone demethylase LDL2. In certain preferred embodiments, the SUVR5 protein complexes with both LDL1 and LDL2.

Recombinant SUVR5-Like Proteins

Certain aspects of the present disclosure relate to recombinant nucleic acids encoding SUVR5-like proteins that contain a heterologous DNA-binding domain of the present disclosure. Examples of heterologous DNA-binding domains include, without limitation, a CCCH zinc finger domain, a multi-cysteine zinc finger domain, a zinc binuclear cluster domain, a C2H2 zinc finger domain having less than three zinc fingers, a C2H2 zinc finger domain having more than three zinc fingers, a zinc finger array, a TAL effector targeting domain, a helix-turn-helix family DNA-binding domain, a basic domain, a ribbon-helix-helix domain, a TBP domain, a barrel dimer domain, a real homology domain, a BAH domain, a SANT domain, a Chromodomain, a Tudor domain, a Bromodomain, a PHD domain, a WD40 domain and a MBD domain.

In one aspect, the present disclosure provides a recombinant nucleic acid encoding an SUVR5-like protein, where the SUVR5-like protein contains a DNA-binding domain, a C-terminal pre-SET domain of the present disclosure, a C-terminal SET domain of the present disclosure, and a C-terminal post-SET domain of the present disclosure, and where the DNA-binding domain is heterologous to an SUVR5 DNA-binding domain. By heterologous, it is meant that the DNA-binding domain does not naturally occur (i.e., is not endogenous) in an SUVR5 protein.

In another aspect, the present disclosure provides a recombinant nucleic acid encoding an SUVR5-like protein, where the SUVR5-like protein contains a DNA-binding domain, a C-terminal pre-SET domain of the present disclosure, a C-terminal SET domain of the present disclosure, and a C-terminal post-SET domain of the present disclosure, and where the DNA-binding domain is not the DNA-binding domain of A. thaliana SUVR5 (i.e., SEQ ID NO: 1).

In yet another aspect, the present disclosure provides a recombinant nucleic acid encoding an SUVR5-like protein, where the SUVR5-like protein contains a DNA-binding domain, a C-terminal pre-SET domain of the present disclosure, a C-terminal SET domain of the present disclosure, and a C-terminal post-SET domain of the present disclosure, and where the DNA-binding domain is any DNA-binding domain other than the DNA-binding domain of SEQ ID NO: 1.

In still another aspect, the present disclosure provides a recombinant nucleic acid encoding an SUVR5-like protein, where the SUVR5-like protein contains a DNA-binding domain, a C-terminal pre-SET domain of the present disclosure, a C-terminal SET domain of the present disclosure, and a C-terminal post-SET domain of the present disclosure, and where the DNA-binding domain contains an amino acid sequence that is less than 99%, less than 98%, less than 97%, less than 96%, less than 95%, less than 94%, less than 93%, less than 92%, less than 91%, less than 90%, less than 85%, less than 80%, less than 75%, less than 70%, less than 65%, less than 60%, less than 55%, or less than 50% identical to SEQ ID NO: 1.

In another aspect, the present disclosure provides a recombinant nucleic acid encoding an SUVR5-like protein, where the SUVR5-like protein contains a DNA-binding domain, a C-terminal pre-SET domain of the present disclosure, a C-terminal SET domain of the present disclosure, and a C-terminal post-SET domain of the present disclosure, and where the DNA-binding domain contains an amino acid sequence with at least one, at least two, at least three, at least four, at least five, at least six, at least seven, at least eight, at least nine, at least 10, or more amino acid differences as compared to the amino acid sequence of SEQ ID NO: 1.

In a further aspect, the present disclosure provides a recombinant nucleic acid encoding an SUVR5-like protein, where the SUVR5-like protein contains a DNA-binding domain, a C-terminal pre-SET domain of the present disclosure, a C-terminal SET domain of the present disclosure, and a C-terminal post-SET domain of the present disclosure, and where the DNA-binding domain binds with a sequence specificity other than that of SEQ ID NO: 1. By a sequence specificity other than that of SEQ ID NO: 1, it is meant that the DNA-binding domain of the SUVR5-like protein has reduced binding affinity to the nucleic acid sequence that is bound by the DNA-binding domain of the A. thaliana SUVR5 protein in comparison to A. thaliana SUVR5 protein. In certain preferred embodiments, the SUVR-like protein contains a DNA-binding domain that has reduced binding affinity or does not bind the nucleic acid sequence: TACTAGTA.

In certain aspects, the present disclosure provides a recombinant nucleic acid encoding an SUVR5-like protein, where the SUVR5-like protein contains more than one DNA-binding domain. In certain aspects, at least one of the DNA-binding domains is the endogenous DNA-binding domain that occurs naturally in a SUVR5 protein. In certain aspects, at least one of the DNA-binding domains is a DNA-binding domain that is heterologous to the SUVR5 DNA-binding domain (i.e., is non-naturally occurring in the SUVR5 protein or is not endogenous in the SUVR5 protein). In certain preferred aspects, SUVR5-like proteins of the present disclosure contain one additional DNA-binding domain in addition to the heterologous DNA-binding domain. In certain preferred embodiments, SUVR5-like proteins of the present disclosure with more than one DNA-binding domain contain both a heterologous DNA-binding domain and an endogenous SUVR5 DNA-binding domain.

### Target Nucleic Acids of the Present Disclosure

Other aspects of the present disclosure relate to utilizing SUVR5 proteins or SUVR5-like proteins to reduce the expression of one or more genes of interest in plants by binding to one or more target nucleic acids associated with the genes of interest. In certain embodiments, SUVR5 proteins or SUVR5-like proteins reduce expression of a gene of interest by binding to a target nucleic acid. In certain preferred embodiments, SUVR5 proteins or SUVR5-like proteins silence expression of a gene of interest by binding to a target nucleic acid.

In certain embodiments, a target nucleic acid of the present disclosure is a nucleic acid that is located at any location within a target gene that provides a suitable location for reducing expression. The target nucleic acid may be located within the coding region of a target gene or upstream or downstream thereof. Moreover, the target nucleic acid may reside endogenously in a target gene or may be inserted into the gene, e.g., heterologous, for example, using techniques such as homologous recombination. For example, a target gene of the present disclosure can be operably linked to a control region, such as a promoter, that contains a sequence that is recognized and bound by an SUVR5 protein or SUVR5-like protein of the present disclosure.

The target nucleic acid may be any given nucleic acid of interest that can be bound by an SUVR5 protein or SUVR5-like protein of the present disclosure. In certain embodiments, the target nucleic acid is endogenous to the plant where the expression of one or more genes is reduced by a SUVR5 protein or SUVR5-like protein of the present disclosure. In other embodiments, the target nucleic acid is a transgene of interest that has been inserted into a plant. Methods of introducing transgenes into plants are well known in the art. Transgenes may be inserted into plants in order to provide a production system for a desired protein, or may be added to the genetic compliment in order to modulate the metabolism of a plant.

Examples of suitable endogenous plant genes whose expression can be reduced by an SUVR5 protein or SUVR5-like protein of the present disclosure include, without limitation, genes that prevent the enhancement of one or more desired traits and genes that prevent increased crop yields. In one non-limiting example, SUVR5 proteins or SUVR5-like proteins of the present disclosure may be used to reduce the expression of the gene GAI in plants, which would create plants that are less sensitive to gibberellin. In embodiments relating to research, SUVR5 proteins or SUVR5-like proteins of the present disclosure may be utilized to silence the expression of an endogenous gene of interest in order to generate mutant plants in which to study the function of the gene of interest.

Examples of suitable transgenes present in plants whose expression can be reduced by an SUVR5 protein or SUVR5-like protein of the present disclosure include, without limitation, transgenes that are not useful in certain genetic backgrounds, transgenes that are harmful in certain genetic backgrounds, and transgenes that are expressed in certain tissues that are undesirable. For example, in the case of transgenes that are expressed in certain tissues that are undesirable, SUVR5 proteins of the present disclosure can be utilized to silence the expression of such transgenes in specific tissues at specific times by operably linking tissue specific promoters to the SUVR5 protein-encoding nucleic acid. In embodiments relating to research, SUVR5 protein of the present disclosure may be utilized to dynamically study transgenes of interest by controlling the induction/silencing of the transgenes.

### Plants of the Present Disclosure

Certain aspects of the present disclosure relate to plants containing one or more recombinant SUVR5 proteins or SUVR5-like proteins of the present disclosure. In certain embodiments, the SUVR5 protein binds to one or more target nucleic acids in the plant and reduces the expression of the one or more target nucleic acids.

As used herein, a “plant” refers to any of various photosynthetic, eukaryotic multi-cellular organisms of the kingdom Plantae, characteristically producing embryos, containing chloroplasts, having cellulose cell walls and lacking locomotion. As used herein, a “plant” includes any plant or part of a plant at any stage of development, including seeds, suspension cultures, plant cells, embryos, meristematic regions, callus tissue, leaves, roots, shoots, gametophytes, sporophytes, pollen, microspores, and progeny thereof. Also included are cuttings, and cell or tissue cultures. As used in conjunction with the present disclosure, plant tissue includes, without limitation, whole plants, plant cells, plant organs, e.g., leafs, stems, roots, meristems, plant seeds, protoplasts, callus, cell cultures, and any groups of plant cells organized into structural and/or functional units.

Any plant cell may be used in the present disclosure so long as it remains viable after being transformed with a sequence of nucleic acids. Preferably, the plant cell is not adversely affected by the transduction of the necessary nucleic acid sequences, the subsequent expression of the proteins or the resulting intermediates.

As disclosed herein, a broad range of plant types may be modified to incorporate an SUVR5 protein or SUVR5-like protein of the present disclosure. Suitable plants that may be modified include both monocotyledonous (monocot) plants and dicotyledonous (dicot) plants.

Examples of suitable plants include, without limitation, species of the Family Gramineae, including Sorghum bicolor and Zea mays; species of the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum.

In certain embodiments, plant cells may include, without limitation, those from corn (Zea mays), canola (Brassica napus, Brassica rapa ssp.), Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), duckweed (Lemna), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia spp.), almond (Prunus amygdalus), sugar beets (Beta vulgaris), sugarcane (Saccharum spp.), oats, barley, vegetables, ornamentals, and conifers.

Examples of suitable vegetables plants include, without limitation, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Examples of suitable ornamental plants include, without limitation, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiapulcherrima), and chrysanthemum.

Examples of suitable conifer plants include, without limitation, loblolly pine (Pinus taeda), slash pine (Pinus elliotii), ponderosa pine (Pinus ponderosa), lodgepole pine (Pinus contorta), Monterey pine (Pinus radiata), Douglas-fir (Pseudotsuga menziesii), Western hemlock (Isuga canadensis), Sitka spruce (Picea glauca), redwood (Sequoia sempervirens), silver fir (Abies amabilis), balsam fir (Abies balsamea), Western red cedar (Thuja plicata), and Alaska yellow-cedar (Chamaecyparis nootkatensis).

Examples of suitable leguminous plants include, without limitation, guar, locust bean, fenugreek, soybean, garden beans, cowpea, mungbean, lima bean, fava bean, lentils, chickpea, peanuts (Arachis sp.), crown vetch (Vicia sp.), hairy vetch, adzuki bean, lupine (Lupinus sp.), trifolium, common bean (Phaseolus sp.), field bean (Pisum sp.), clover (Melilotus sp.) Lotus, trefoil, lens, and false indigo.

Examples of suitable forage and turf grass include, without limitation, alfalfa (Medicago s sp.), orchard grass, tall fescue, perennial ryegrass, creeping bent grass, and redtop.

Examples of suitable crop plants and model plants include, without limitation, Arabidopsis, corn, rice, alfalfa, sunflower, canola, soybean, cotton, peanut, sorghum, wheat, tobacco, and lemna.

The plants of the present disclosure may be genetically modified in that recombinant nucleic acids have been introduced into the plants, and as such the genetically modified plants do not occur in nature. A suitable plant of the present disclosure is one capable of expressing one or more nucleic acid constructs encoding one or more SUVR5 proteins or SUVR5-like proteins of the present disclosure.

As used herein, the terms “transgenic plant” and “genetically modified plant” are used interchangeably and refer to a plant which contains within its genome a recombinant nucleic acid. Generally, the recombinant nucleic acid is stably integrated within the genome such that the polynucleotide is passed on to successive generations. However, in certain embodiments, the recombinant nucleic acid is transiently expressed in the plant. The recombinant nucleic acid may be integrated into the genome alone or as part of a recombinant expression cassette. “Transgenic” is used herein to include any cell, cell line, callus, tissue, plant part or plant, the genotype of which has been altered by the presence of exogenous nucleic acid including those transgenics initially so altered as well as those created by sexual crosses or asexual propagation from the initial transgenic.

“Recombinant nucleic acid” or “heterologous nucleic acid” or “recombinant polynucleotide” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids contains two or more subsequences that are not found in the same relationship to each other in nature. For example, regarding instance (c), a recombinant nucleic acid sequence will have two or more sequences from unrelated genes arranged to make a new functional nucleic acid. Specifically, the present disclosure describes the introduction of an expression vector into a plant cell, where the expression vector contains a nucleic acid sequence coding for a protein that is not normally found in a plant cell or contains a nucleic acid coding for a protein that is normally found in a plant cell but is under the control of different regulatory sequences. With reference to the plant cell's genome, then, the nucleic acid sequence that codes for the protein is recombinant. A protein that is referred to as recombinant generally implies that it is encoded by a recombinant nucleic acid sequence in the plant cell.

A “recombinant” polypeptide, protein, or enzyme of the present disclosure, is a polypeptide, protein, or enzyme that is encoded by a “recombinant nucleic acid” or “heterologous nucleic acid” or “recombinant polynucleotide.”

In some embodiments, the genes encoding the desired proteins in the plant cell may be heterologous to the plant cell or these genes may be endogenous to the host cell but are operatively linked to heterologous promoters and/or control regions which result in the higher expression of the gene(s) in the plant cell. In certain embodiments, the plant cell does not naturally produce the desired proteins, and contains heterologous nucleic acid constructs capable of expressing one or more genes necessary for producing those molecules.

Expression of SUVR5 Proteins in Plants

SUVR5 proteins or SUVR5-like protein of the present disclosure may be introduced into plant cells via any suitable methods known in the art. For example, the SUVR5 protein or SUVR5-like protein can be exogenously added to plant cells and the plant cells are maintained under conditions such that the SUVR5 protein binds to one or more target nucleic acids and reduces the expression of the target nucleic acids in the plant cells. Alternatively, a recombinant nucleic acid encoding an SUVR5 protein or SUVR5-like protein of the present disclosure can be expressed in plant cells and the plant cells are maintained under conditions such that the expressed SUVR5 protein or SUVR-like protein binds to one or more target nucleic acids and reduces the expression of the target gene in the plant cells. Additionally, in certain embodiments, an SUVR5 protein or SUVR5-like protein of the present disclosure may be transiently expressed in a plant via viral infection of the plant, or by introducing an SUVR5 protein-encoding RNA into a plant to temporarily reduce or silence the expression of a gene of interest. Methods of introducing recombinant proteins via viral infection or via the introduction of RNAs into plants are well known in the art.

A recombinant nucleic acid encoding an SUVR5 protein or SUVR5-like protein of the present disclosure can be expressed in a plant with any suitable plant expression vector. Typical vectors useful for expression of recombinant nucleic acids in higher plants are well known in the art and include, without limitation, vectors derived from the tumor-inducing (Ti) plasmid of Agrobacterium tumefaciens (e.g., see Rogers et al., Meth. in Enzymol. (1987) 153:253-277). These vectors are plant integrating vectors in that on transformation, the vectors integrate a portion of vector DNA into the genome of the host plant. Exemplary A. tumefaciens vectors useful herein are plasmids pKYLX6 and pKYLX7 (e.g., see of Schardl et al., Gene (1987) 61:1-11; and Berger et al., Proc. Natl. Acad. Sci. USA (1989) 86:8402-8406); and plasmid pBI 101.2 that is available from Clontech Laboratories, Inc. (Palo Alto, Calif.).

In addition to regulatory domains, an SUVR5 protein or SUVR5-like protein of the present disclosure can be expressed as a fusion protein that is coupled to, for example, a maltose binding protein (“MBP”), glutathione S transferase (GST), hexahistidine, c-myc, or the FLAG epitope for ease of purification, monitoring expression, or monitoring cellular and subcellular localization.

Moreover, a recombinant nucleic acid encoding an SUVR5 protein or SUVR5-like protein of the present disclosure can be modified to improve expression of the SUVR5 protein or SUVR5-like protein in plants by using codon preference. When the recombinant nucleic acid is prepared or altered synthetically, advantage can be taken of known codon preferences of the intended plant host where the nucleic acid is to be expressed. For example, recombinant nucleic acids of the present disclosure can be modified to account for the specific codon preferences and GC content preferences of monocotyledons and dicotyledons, as these preferences have been shown to differ (Murray et al., Nucl. Acids Res. (1989) 17: 477-498).

In some embodiments, SUVR5 proteins or SUVR5-like proteins of the present disclosure can be used to create functional “gene knockout” mutations in a plant by repression of the target gene expression. Repression may be of a structural gene, e.g., one encoding a protein having for example enzymatic activity, or of a regulatory gene, e.g., one encoding a protein that in turn regulates expression of a structural gene.

The present disclosure further provides expression vectors containing a recombinant SUVR5 protein-encoding nucleic acid or SUVR5-like protein-encoding nucleic acid of the present disclosure. A nucleic acid sequence coding for the desired recombinant nucleic acid of the present disclosure can be used to construct a recombinant expression vector which can be introduced into the desired host cell. A recombinant expression vector will typically contain a nucleic acid encoding an SUVR5 protein or SUVR5-like protein of the present disclosure, operably linked to transcriptional initiation regulatory sequences which will direct the transcription of the nucleic acid in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A plant promoter, or functional fragment thereof, can be employed to control the expression of a recombinant nucleic acid of the present disclosure in regenerated plants. The selection of the promoter used in expression vectors will determine the spatial and temporal expression pattern of the recombinant nucleic acid in the modified plant, e.g., the recombinant SUVR5-encoding nucleic acid is only expressed in the desired tissue or at a certain time in plant development or growth. Certain promoters will express recombinant nucleic acids in all plant tissues and are active under most environmental conditions and states of development or cell differentiation (i.e., constitutive promoters). Other promoters will express recombinant nucleic acids in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the recombinant nucleic acid under various inducing conditions.

Examples of suitable constitutive promoters include, without limitation, the core promoter of the Rsyn7, the core CaMV 35S promoter (Odell et al., Nature (1985) 313:810-812), CaMV 19S (Lawton et al., 1987), rice actin (Wang et al., 1992; U.S. Pat. No. 5,641,876; and McElroy et al., Plant Cell (1985) 2:163-171); ubiquitin (Christensen et al., Plant Mol. Biol. (1989)12:619-632; and Christensen et al., Plant Mol. Biol. (1992) 18:675-689), pEMU (Last et al., Theor. Appl. Genet. (1991) 81:581-588), MAS (Velten et al., EMBO J. (1984) 3:2723-2730), nos (Ebert et al., 1987), Adh (Walker et al., 1987), the P- or 2′-promoter derived from T-DNA of Agrobacterium tumefaciens, the Smas promoter, the cinnamyl alcohol dehydrogenase promoter (U.S. Pat. No. 5,683,439), the Nos promoter, the pEmu promoter, the rubisco promoter, the GRP 1-8 promoter, and other transcription initiation regions from various plant genes known to those of skilled artisans, and constitutive promoters described in, for example, U.S. Pat. Nos. 5,608,149; 5,608,144; 5,604,121; 5,569,597; 5,466,785; 5,399,680; 5,268,463; and 5,608,142.

Examples of suitable tissue specific promoters include, without limitation, the lectin promoter (Vodkin et al., 1983; Lindstrom et al., 1990), the corn alcohol dehydrogenase 1 promoter (Vogel et al., 1989; Dennis et al., 1984), the corn light harvesting complex promoter (Simpson, 1986; Bansal et al., 1992), the corn heat shock protein promoter (Odell et al., Nature (1985) 313:810-812; Rochester et al., 1986), the pea small subunit RuBP carboxylase promoter (Poulsen et al., 1986; Cashmore et al., 1983), the Ti plasmid mannopine synthase promoter (Langridge et al., 1989), the Ti plasmid nopaline synthase promoter (Langridge et al., 1989), the petunia chalcone isomerase promoter (Van Tunen et al., 1988), the bean glycine rich protein 1 promoter (Keller et al., 1989), the truncated CaMV 35s promoter (Odell et al., Nature (1985) 313:810-812), the potato patatin promoter (Wenzler et al., 1989), the root cell promoter (Conkling et al., 1990), the maize zein promoter (Reina et al., 1990; Kriz et al., 1987; Wandelt and Feix, 1989; Langridge and Feix, 1983; Reina et al., 1990), the globulin-1 promoter (Belanger and Kriz et al., 1991), the α-tubulin promoter, the cab promoter (Sullivan et al., 1989), the PEPCase promoter (Hudspeth & Grula, 1989), the R gene complex-associated promoters (Chandler et al., 1989), and the chalcone synthase promoters (Franken et al., 1991).

Alternatively, the plant promoter can direct expression of a recombinant nucleic acid of the present disclosure in a specific tissue or may be otherwise under more precise environmental or developmental control. Such promoters are referred to here as “inducible” promoters. Environmental conditions that may affect transcription by inducible promoters include, without limitation, pathogen attack, anaerobic conditions, or the presence of light. Examples of inducible promoters include, without limitation, the AdhI promoter which is inducible by hypoxia or cold stress, the Hsp70 promoter which is inducible by heat stress, and the PPDK promoter which is inducible by light. Examples of promoters under developmental control include, without limitation, promoters that initiate transcription only, or preferentially, in certain tissues, such as leaves, roots, fruit, seeds, or flowers. An exemplary promoter is the anther specific promoter 5126 (U.S. Pat. Nos. 5,689,049 and 5,689,051). The operation of a promoter may also vary depending on its location in the genome. Thus, an inducible promoter may become fully or partially constitutive in certain locations.

Moreover, any combination of a constitutive or inducible promoter, and a non-tissue specific or tissue specific promoter may be used to control the expression of an SUVR5 protein or SUVR5-like protein of the present disclosure.

Both heterologous and endogenous promoters can be employed to direct expression of recombinant nucleic acids of the present disclosure. Accordingly, in certain embodiments, expression of a recombinant SUVR5-encoding nucleic acid or SUVR5-like protein-encoding nucleic acid of the present disclosure is under the control of its endogenous promoter. In other embodiments, expression of a recombinant SUVR5-encoding nucleic acid or SUVR5-like protein-encoding nucleic acid of the present disclosure is under the control of a heterologous promoter. Additionally, an endogenous SUVR5 gene of the present disclosure can be modified using a knock-in approach, so that the modified SUVR5 gene will be under the control of its endogenous elements. Alternatively, a modified form of an entire SURV5 genomic sequence may be introduced into a plant, so that the modified gene will be under the control of its endogenous elements and the wild-type SUVR5 gene remains intact. Any or all of these techniques may also be combined to direct the expression of a recombinant nucleic acid of the present disclosure.

In other embodiments, isolated nucleic acids which serve as promoter or enhancer elements can be introduced in the appropriate position (generally upstream) of an endogenous form of an SUVR5-encoding nucleic acid of the present disclosure so as to up or down regulate expression of the SUVR5-encoding nucleic acid. For example, endogenous promoters can be altered in vivo by mutation, deletion, and/or substitution (e.g., see U.S. Pat. No. 5,565,350; and PCT/US93/03868), or isolated promoters can be introduced into a plant cell in the proper orientation and distance from an SUVR5-encoding nucleic acid of the present disclosure so as to control the expression of the SUVR5-encoding nucleic acid. Expression can be modulated under conditions suitable for plant growth so as to alter the total concentration and/or alter the composition of the SUVR5 proteins of the present disclosure in a plant cell.

Plant transformation protocols as well as protocols for introducing recombinant nucleic acids of the present disclosure into plants may vary depending on the type of plant or plant cell, e.g., monocot or dicot, targeted for transformation. Suitable methods of introducing recombinant nucleic acids of the present disclosure into plant cells and subsequent insertion into the plant genome include, without limitation, microinjection (Crossway et al., Biotechniques (1986) 4:320-334), electroporation (Riggs et al., Proc. Natl. Acad Sci. USA (1986) 83:5602-5606), Agrobacterium-mediated transformation (U.S. Pat. No. 5,563,055), direct gene transfer (Paszkowski et al., EMBO J. (1984) 3:2717-2722), and ballistic particle acceleration (U.S. Pat. No. 4,945,050; Tomes et al. (1995). “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al., Biotechnology (1988) 6:923-926).

Additionally, SUVR5 proteins or SUVR5-like proteins of the present disclosure can be targeted to a specific organelle within a plant cell. Targeting can be achieved by providing the SUVR5 protein or SUVR5-like protein with an appropriate targeting peptide sequence. Examples of such targeting peptides include, without limitation, secretory signal peptides (for secretion or cell wall or membrane targeting), plastid transit peptides, chloroplast transit peptides, mitochondrial target peptides, vacuole targeting peptides, nuclear targeting peptides, and the like (e.g., see Reiss et al., Mol. Gen. Genet. (1987) 209(1):116-121; Settles and Martienssen, Trends Cell Biol (1998) 12:494-501; Scott et al., J Biol Chem (2000) 10:1074; and Luque and Correas, J Cell Sci (2000) 113:2485-2495).

The modified plant may be grown into plants in accordance with conventional ways (e.g., see McCormick et al., Plant Cell. Reports (1986) 81-84.). These plants may then be grown, and pollinated with either the same transformed strain or different strains, with the resulting hybrid having the desired phenotypic characteristic. Two or more generations may be grown to ensure that the subject phenotypic characteristic is stably maintained and inherited and then seeds harvested to ensure the desired phenotype or other property has been achieved.

### Methods of Reducing Gene Expression in Plants

Further aspects of the present disclosure relate to methods for reducing expression of one or more target nucleic acids, such as genes, in a plant by utilizing SUVR5 proteins or SUVR5-like protein of the present disclosure. In one aspect, the present disclosure provides a method for reducing expression of one or more target nucleic acids in a plant, by providing a plant containing a recombinant polypeptide of the present disclosure, such as an SUVR5 protein, where the recombinant polypeptide contains a DNA-binding domain of the present disclosure, a C-terminal pre-SET domain of the present disclosure, a C-terminal SET domain of the present disclosure, and a C-terminal post-SET domain of the present disclosure; and growing the plant under conditions whereby the recombinant polypeptide binds to one or more target nucleic acids of the present disclosure, thereby reducing expression of the one or more target genes. Any plant described herein and containing a recombinant polypeptide of the present disclosure may be used. In certain embodiments, the recombinant polypeptide is an SUVR5 protein that contains a heterologous DNA-binding domain, such as a TAL effector targeting domain or an engineered zinc finger domain.

Growing conditions sufficient for the recombinant polypeptide expressed in the plant to bind to and reduce the expression of one or more target nucleic acids of the present disclosure are well known in the art and include any suitable growing conditions disclosed herein. Typically, the plant is grown under conditions sufficient to express the recombinant polypeptide, such as an SUVR5 protein or SUVR5-like protein of the present disclosure, and for the expressed recombinant polypeptide to be localized to the nucleus of cells of the plant in order to bind to and reduce the expression of the target nucleic acids. Generally, the conditions sufficient for the expression of the recombinant polypeptide will depend on the promoter used to control the expression of the recombinant polypeptide. For example, if an inducible promoter is utilized, expression of the recombinant polypeptide in a plant will require that the plant to be grown in the presence of the inducer.

It is to be understood that while the present disclosure has been described in conjunction with the preferred specific embodiments thereof, the foregoing description is intended to illustrate and not limit the scope of the present disclosure. Other aspects, advantages, and modifications within the scope of the present disclosure will be apparent to those skilled in the art to which the present disclosure pertains.

The following examples are offered to illustrate provided embodiments and are not intended to limit the scope of the present disclosure.

### EXAMPLES

### Example 1

The following Example relates to the characterization of the Arabidopsis thaliana protein SUVR5 as a repressor of gene expression.

### Introduction

In eukaryotes, chromatin structure regulates the access of transcriptional machinery to gene regulatory sequences, playing an important role in gene regulation and genome stability. The transition between transcriptionally active and transcriptionally repressed chromatin states is controlled by covalent modifications of the histone tails, methylation of cytosines in the DNA that is wrapped around the histones, and the differential use of histone variants [1]. In mammals and plants, heterochromatin is associated with cytosine methylation and histone tail modifications such as methylation of H3 at lysine 9 (H3K9me).

In plants, DNA methylation occurs in three different contexts, CG, CHG and CHH, and in all cases, de novo DNA methylation is established by DRM2 (DOMAINS REARRANGED METHYLTRANSFERASE 2), a homolog of the DNMT3 family. Then, each methylation context is maintained through the cell cycle for the perpetuation of the heterochromatinic state by different pathways that use different DNA methyl transferases [2].

DRM2 is also responsible for the maintenance of CHH methylation through persistent de novo methylation [2,3].

MET1 (DNA METHYLTRANSFERASE 1), a homolog of DNMT1, maintains CG methylation with the aid of the VIM/ORTH family [4,5,6] whose members are homologs of the mammalian UHRF1, and some of whose SRA domains have been shown to bind hemimethylated CG sites [7], supporting the current model in which VIM/UHRF1 proteins would recruit MET1/DNMT1 to sites of hemimethylated DNA after S phase of the cell cycle to allow the restoration to the fully methylated state and the preservation of the heterochromatinic state [8,9].

CHROMOMETHYLASE 3 (CMT3), a plant specific methyltransferase, is responsible for CHG methylation, while other three SRA domain proteins (SUVH4/KRYPTONITE, SUVH5, and SUVH6) are also required to maintain it [10,11,12,13]. SUVH4, SUVH5 and SUVH6 are histone lysine methyl transferases (HKMTases, chromatin modifying enzymes involved in the establishment and/or maintenance of the silent chromatin state by catalyzing the transfer of methyl groups to the lysine 9 residue of histone H3 protein). Their catalytic activity resides in the SET (Suppressor of variegation, Enhancer of zeste and Trithorax) domain. SUVH4/KYP, SUVH5 and SUVH6 have been shown to have in vitro H3K9 HMT activity [12], and for SUVH4/KYP its activity has also been confirmed by mass spectrometric analysis of in vitro methylated histones [10,11,14]. The repressive state of chromatin induced by histone lysine 9 methylation has been shown to be mechanistically linked to DNA methylation in Arabidopsis. Mutations in SUVH4/KYP result in decreased H3K9me2 and decreased cytosine methylation [10,15,16] which are even further reduced in suvh4 suvh5, suvh4 suvh6 and suvh4 suvh5 suvh6 triple mutants at specific genomic loci [12,13], whereas a loss of DNA methylation in met1 correlates with a loss of H3K9me2 [16]. Moreover, there seems to be a clear genome-wide correlation between the heterochromatinic H3K9me2 and DNA methylation [17].

This correlation can be explained by the methylated-DNA binding ability of the SRA domains of SUVH4, SUVH5 and SUVH6, with SUVH4 strongly preferring CHG methylation, SUVH5 binding to methylation in all contexts and SUVH6 preferring both CHG and CHH methylation strongly over CG methylation [7,18]. The structure of the SUVH5 SRA domain bound to methylated DNA has even been reported, showing that, unlike the UHRF1 SRA domain or the MBD domains of MBD1 and MeCP2 where a single domain recognizes both strands, in the SUVH5 complex, two SRA domains bind independently to each strand of the DNA duplex at either a fully or hemimethylated site [19].

These results support a model where regions rich in DNA methylation may attract SUVH4, SUVH5, and/or SUVH6, leading to H3K9 methylation. Histone methylation would then provide a binding site for CMT3 leading to CHG methylation, and thus creating a self-reinforcing feedback loop for the maintenance of DNA and histone methylation able to explain the stability of epigenetic silent states and their self-perpetuating nature [7].

Thus far, H3K9me2 repression has been linked to DNA methylation in purely epigenetic feedback loops. The following Example shows that Arabidopsis thaliana SUVR5 is able to recognize specific DNA sequences and start heterochromatin nucleation at those loci through DNA-methylation independent H3K9me2 deposition, acting as part of a multimeric complex that includes other histone tail modifying activities. Without wishing to be bound by theory, it is believed that the SUVR5 mechanism of action is an example of heterochromatin formation that stands apart from the self-perpetuating loop between H3K9me2 and DNA methylation, and that it allows for the increased plasticity needed in the response to environmental or developmental cues during the organism life.

### Materials and Methods

Plant Strains

The wild-type control in this study was the Columbia 0 ecotype. suvr5-1 (Joshua S. Mylne, Lynne Barrett, Federico Tessadori, Stéphane Mesnage, Lianna Johnson, Yana V. Bernatavichute, Steven E. Jacobsen, Paul Fransz and Caroline Dean. (2006) LHP1, the Arabidopsis homologue of HETEROCHROMATIN PROTEIN1, is required for epigenetic silencing of FLC. Proc. Nat. Acad. Sci. U.S.A. 103: 5012-5017) and suvr5-2 are T-DNA insertion lines obtained from the SALK Institute Genomic Analysis Laboratory (SALK_{—}026224 and SALK_{—}085717 respectively). The suvh4 suvh5 suvh6 line was described in [37]. The ldl1-2 ldl2 line was described in [38].

Alignments

The identification of SUVR5 plant homologs, their sequences and their alignment was obtained from Phytozome.

ChIP

For the ChIP experiments of H3K9me2, a previously described protocol was used in 3 week old leaves of wild type Col0 and suvr5-1 plants [39].

The ChIP-chip was performed as described in [17], the results show a comparison of the abundance of DNA pulled down with the anti-H3K9me2 antibody (#1220, monoclonal anti-H3K9m2 antibody, Abcam) versus INPUT.

For validation of the ChIP-chip results, qPCR was done using the primers listed in Table 2.

TABLE 2

| PRIMER NAME | SEQUENCE | GENE |
|---|---|---|
| JP2454 | TCTCTCTCGCTGCTTCTCG | ACT7 |
| (SEQ ID NO: 39) | | |
| JP2455 | GCAAAATCAAGCGAACGG | ACT7 |
| (SEQ ID NO: 40) | | |
| JP9836 | GTGGCCGTGATCGGACTA | AT1G12160 |
| (SEQ ID NO: 41) | | |
| JP9837 | CAACGCTAACCGAGTCTGAA | AT1G12160 |
| (SEQ ID NO: 42) | | |
| JP9842 | GGTCGTGGCTTTGTTCAAGATA | AT1G31290 |
| (SEQ ID NO: 43) | | |
| JP9843 | GCCTTGACTCACTTGAGCTTG | AT1G31290 |
| (SEQ ID NO: 44) | | |
| JP9838 | CGGTGTTACAACTGGTGGAGT | AT3G22121 |
| (SEQ ID NO: 45) | | |
| JP9839 | CAAAACCTCCCATCGTAAAGC | AT3G22121 |
| (SEQ ID NO: 46) | | |
| JP9787 | TCGACTTGTTTGGACCTTGA | AT4G36510 |
| (SEQ ID NO: 47) | | |
| JP9788 | TCATGCGAATTATAGAAATTTAGACC | AT4G36510 |
| (SEQ ID NO: 48) | | |

H3K9me2 ChIP-chip Analysis

Each probe in the array was normalized by taking the log 2 ratio of H3K9m2 to INPUT intensities, and the scores were scaled so that the average score across the arrays were zero. H3K9m2 hypomethylated regions were defined by tiling the genome into 500 bp bins (250 bp overlap), and computing the log 2 ratios of the scores of suvr5 vs. Col, and Z-score transformed. A Z<−3 cutoff was applied, and regions within 2.5 kb were merged.

RT-qPCR

RNA was extracted from 0.2 g of tissue using Trizol (Invitrogen) and following the manufacturer's instructions. 1 μg of total RNA was used for RT-PCR using SuperScript III (Invitrogen). qPCR was performed using iQ SYBR Green Supermix (#170-8880, BioRad). Three biological replicas were sampled and standard deviations determined. The primers used were designed using QuantPrime qPCR primer design tool, and are listed in Table 3.

TABLE 3

| PRIMER NAME | SEQUENCE | GENE |
|---|---|---|
| JP2452 | TCGTGGTGGTGAGTTTGTTAC | ACT7 |
| (SEQ ID NO: 49) | | |
| JP2453 | CAGCATCATCACAAGCATCC | ACT7 |
| (SEQ ID NO: 50) | | |
| JP9693 | AGAAATCTTCGACGCGGTCGTG | AT1G12160 |
| (SEQ ID NO: 51) | | |
| JP9694 | TCCCAGGAATATGAGCAAGACGAG | AT1G12160 |
| (SEQ ID NO: 52) | | |
| JP9721 | TCTCACACCGCTAGTGGTTCTC | AT1G31290 |
| (SEQ ID NO: 53) | | |
| JP9722 | TCAGGACGCTTTACTGGTTCTTTC | AT1G31290 |
| (SEQ ID NO: 54) | | |
| JP9709 | CGGTTGGTGGTTTAGGATGGGTAG | AT3G22121 |
| (SEQ ID NO: 55) | | |
| JP9710 | TCTCCTATGCTTGCGACTGTACC | AT3G22121 |
| (SEQ ID NO: 56) | | |
| JP9864 | GCTGTTTGAGTTCGCCGCCC | AT4G36510 |
| (SEQ ID NO: 57) | | |
| JP9865 | CCGACCAAAACTCCACCCGCC | AT4G36510 |
| (SEQ ID NO: 58) | | |
| JP9816 | TTCCGATTCACAGCGACCTAGC | AT3G12830 |
| (SEQ ID NO: 59) | | |
| JP9817 | TTGCTTCTTTGAGCGGCGAGTC | AT3G12830 |
| (SEQ ID NO: 60) | | |
| JP9949 | GCAAAGGGTTCGAGCTTCTTATGG | AT5G54490 |
| (SEQ ID NO: 61) | | |
| JP9950 | CGTCGATGCGTTTCTTCGTAAGC | AT5G54490 |
| (SEQ ID NO: 62) | | |
| JP9965 | GTTGTCACAAATTTCGCTGGCTTG | AT5G13320 |
| (SEQ ID NO: 63) | | |
| JP9966 | GCGCGTTGTTGTAGAAACCAGTC | AT5G13320 |
| (SEQ ID NO: 64) | | |

mRNA-seq

Leaves from 3 week old wild-type Col0, suv5-1 mutant, ldl1-2 ldl2 double mutant, and suvr5-1 ldl1-2 ldl2 triple mutant plants were used for RNA extraction using Trizol (Invitrogen) following the manufacturer instructions. 10 μg of total RNA was treated with DNaseI (Roche), and cleaned up with RNeasy columns (Qiagen). Poly(A) was purified using the Dynabeads mRNA Purification Kit (Invitrogen) and used to generate the mRNA-seq libraries following manufacturer instructions (Illumina). The libraries were sequenced using an Illumina Genome Analyzer.

Gene and transposon expression in the RNA-seq data was measured by calculating reads per kilobase per million mapped reads (RPKM). P-values to detect differential expression were calculated by Fisher's exact test and Benjamini-Hochberg corrected for multiple testing. Genes differentially expressed in wild-type and mutants were defined as those that have log 2(suvr5/wild-type)>4 and P<0.01.

Recombinant Protein Purification

The GST fusion protein used for SELEX and EMSA experiments was made by cloning SUVR5 zinc fingers domain (amino acids 720 to 866) using the Gateway cloning system with pDEST15 as the final destination vector. Protein expression and purification was performed as previously described [7] plus the addition of 100 μM ZnSO₄ to the cell culture after protein expression induction and avoiding the use of EDTA during the protein purification.

SELEX

The basic protocol for SELEX experiments described in [41] was followed with some minor modifications. For the SELEX experiments, 5 μg of a primer with 15 random nucleotides between two adaptor sequences (JP7666: GTT TTC CCA GTC ACT ACN NNN NNN NNN NNN NNG TCA TAG CTG TTT CCT G (SEQ ID NO: 65)) was annealed with 5 μg of the reverse adaptor primer (JP7668: CAG GAA ACA GCT ATG AC (SEQ ID NO: 66)) by boiling and letting them cool down slowly. Then, 1 μg of the annealed primers was used to make dsDNA using Klenow fragment, followed by a standard phenol DNA extraction and resuspension in 200 μL of SELEX binding buffer (25 mM HEPES pH7.5, 50 mM KCl, 2.5 mM MgCl₂, 0.1% NP40, 1 μM ZnSO₄, 5% glycerol).

The purified and glutathione beads-bound GST-SUVR5 zinc fingers domain was incubated with the dsDNA in SELEX buffer, 5 μg of BSA and 5 μg of salmon sperm DNA for 30 minutes at RT. The beads were washed 5 times with 1 mL of SELEX binding buffer followed by a Phenol/Chloroform/IAA DNA extraction and precipitation. The recovered DNA was resuspended 10 μL of TE buffer and used for PCR as follows: (95° C. for 3 min), (95° C. for 30 sec; 60° C. for 1 min; 72° C. for 30 sec)×10 cycles, (72° C. for 10 min). The result of the PCR was used as starting point for the next binding/eluting cycle.

For the standard SELEX experiment, 10 cycles of binding/eluting were done before TOPO ligating the recovered DNA to pCR2.1 vector (Invitrogen) and transforming E. coli TOP10 bacteria (Invitrogen). 20 colonies were sequenced that data was used to find the consensus binding motif using the MEME Suite [42].

For the genomic-SELEX experiment, Arabidopsis thaliana genomic DNA was extracted from wild type 3 week old plant leaves and fragmented to 100 bp using COVARIS. 2 μg of this DNA was processed for end repair and adaptor ligation following manufacturer instructions (Illumina) and used as indicated above for incubation with the glutathione beads-bound GST-SUVR5 zinc fingers domain purified protein. Two genomic-SELEX experiments were done, one in which only one binding/eluting cycle was performed (x1: control) and one were 9 cycles were performed (x9). The recovered DNA was sequenced using an Illumina Genome Analyzer and a random thousand reads were used to identify a binding motif sequence using the analysis tool MEME Suite [42].

EMSA

The protocol described in [7] was followed with slight modifications to the binding buffer (12% glycerol, 20 mM Tris-HCl pH7.5, 50 mM KCl, 1 mM MgCl₂, 1 mM DTT). The primers used to test protein binding are listed in Table 4.

TABLE 4

| PRIMER NAME | SEQUENCE | PROBE |
|---|---|---|
| JP8487 | ACCAAGCAACACACCCCGT | UNSPECIFIC |
| (SEQ ID NO: 67) | | |
| JP8493 | ACGGGGTGTGTTGCTTGGT | UNSPECIFIC |
| (SEQ ID NO: 68) | | |
| JP8489 | GTAGAATACTAGTTGATAAC | SPECIFIC |
| (SEQ ID NO: 69) | | |
| JP8495 | GTTATCAACTAGTATTCTAC | SPECIFIC |
| (SEQ ID NO: 70) | | |
| JP8490 | GTAGAACACTAGTTGATAAC | SPECIFIC PROBE |
| (SEQ ID NO: 71) | | MUTANT 1 |
| JP8496 | GTTATCAACTAGTGTTCTAC | SPECIFIC PROBE |
| (SEQ ID NO: 72) | | MUTANT 1 |
| JP8491 | GTAGAATCCTAGTTGATAAC | SPECIFIC PROBE |
| (SEQ ID NO: 73) | | MUTANT 2 |
| JP8497 | GTTATCAACTAGGATTCTAC | SPECIFIC PROBE |
| (SEQ ID NO: 74) | | MUTANT 2 |
| JP8492 | GTAGAATAATAGTTGATAAC | SPECIFIC PROBE |
| (SEQ ID NO: 75) | | MUTANT 3 |
| JP8498 | GTTATCAACTATTATTCTAC | SPECIFIC PROBE |
| (SEQ ID NO: 76) | | MUTANT 3 |

IP/Mass Spec

Affinity purification of LDL1-3× FLAG was performed as described in [19] with the following modifications: ˜15 grams of inflorescence tissue from transgenic and untransformed (Col-0) plants was ground in liquid nitrogen, and resuspended in 75 ml of lysis buffer (50 mM Tris pH 7.5, 300 mM NaCl, 5 mM MgCl2, 5% glycerol v/v 0.02% NP-40 v/v, 0.5 mM DTT, 1 mg/mL pepstatin, 1 mM PMSF and 1 protease inhibitor cocktail tablet (Roche, 14696200)).

Mass spectrometry analyses were performed as described in [19].

Auxin Treatment

Wild type Col0, suvr5-1 and suvr5-2 plants were either grown for 13 days in vertical MS plates (CONTROL) or grown in vertical MS plates for 6 days before being transferred to MS+0.5 μM NAA (Sigma) plates and let to grow for 7 additional days. Root length was measured at different time points and whole seedlings from both experiments were collected on day 13 and frozen for RNA extraction.

### Results

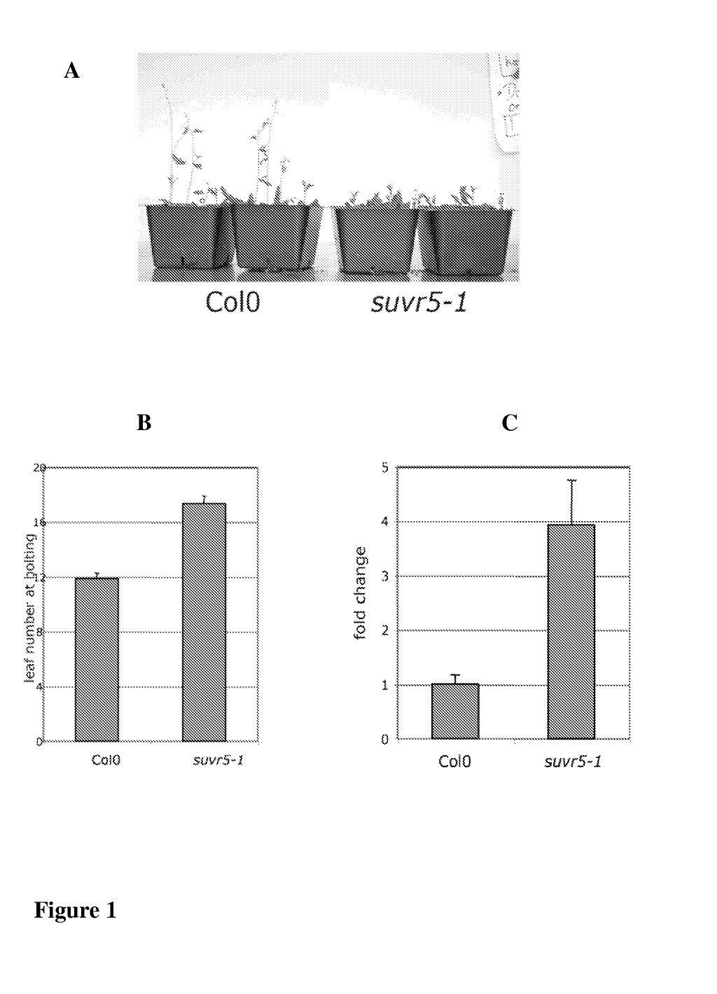

Flowering time is a developmental trait controlled by the expression level of a set of genes that are affected by environmental conditions. One known mechanism of controlling this expression level involves epigenetic modifications such as DNA and histone methylation. Moreover, assaying for early or late flowering phenotypes has, thus far, proven a successful way of screening for factors involved in epigenetic pathways. In an attempt to analyze the involvement of Arabidopsis SU(VAR)3-9 Related family of SET domain-containing proteins in histone methylation, all five known suvr mutants were screened for alterations in flowering time. It was found that the suvr5 mutation produced a delay in flowering time specific to the mutation, and was no longer amplified in the quintuple suvr1 suvr2 suvr3 suvr4 suvr5 mutant. Previous results have shown that SUVR5 is not involved in the vernalization-induced H3K9 methylation at the flowering time controlling gene FLC [23], but recent experiments have allowed the detection of increased levels of FLC transcript in suvr5 mutant non-vernalized adult plants that may account for the observed late flowering phenotype (FIG. 1), which was also previously reported [20].

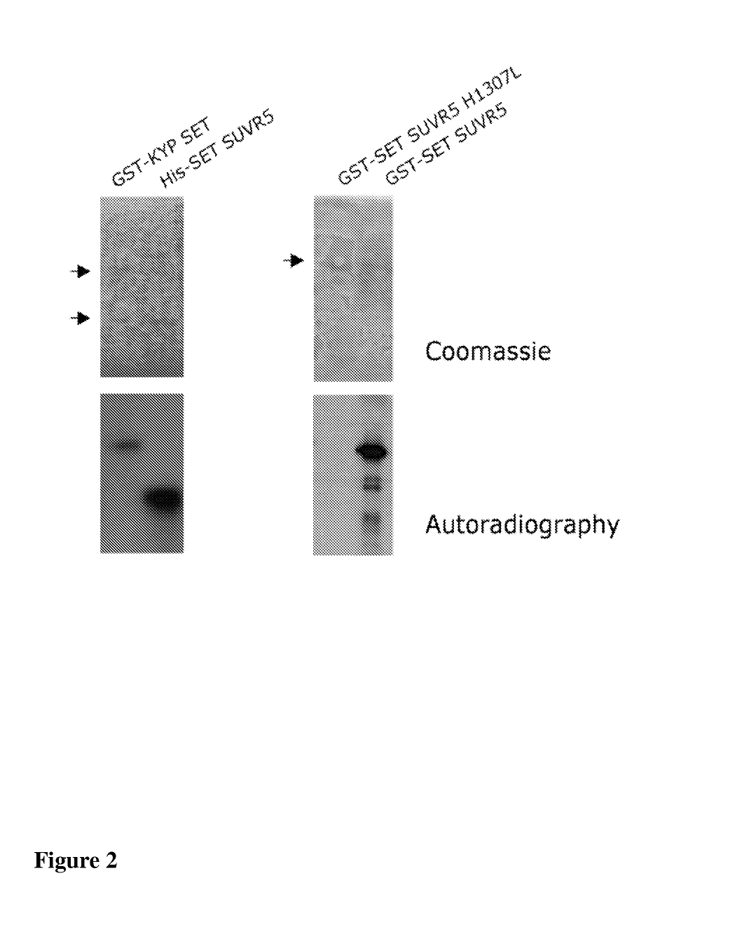

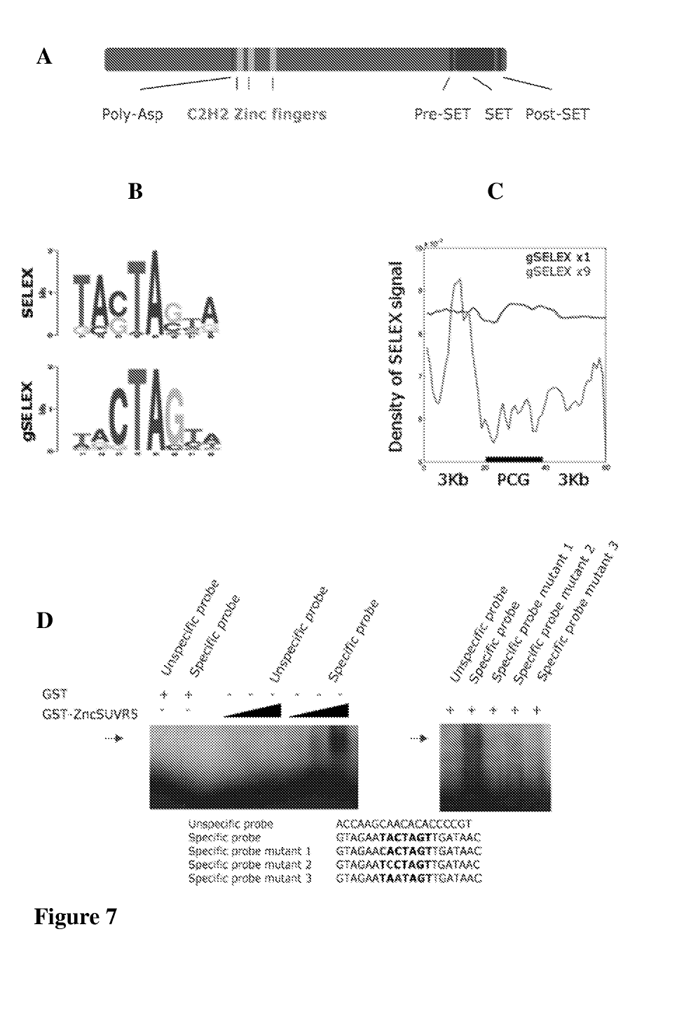

A. thaliana SUVR5 (AtSUVR5) is a member of the SU(VAR)3-9 Related family, with a domain structure that includes a SET domain in the C terminus (i.e., the domain responsible for the catalytic activity of all histone methyltransferases) and a zinc finger domain containing three C2H2 zinc fingers in tandem in the central part of the protein [22] (FIG. 7A). The fact that SUVR5 binds the methyl group donor S-Adenosyl methionine (FIG. 2) and that SUVR5 contains all conserved residues in the HΦΦNHSC motif of the SET domain, which is crucial for HMTase activity, suggest a role for the protein in histone methylation. While, in vitro activity has not been shown, it is believed that this may be due to the need for other cofactors or to the presence of a larger complex.

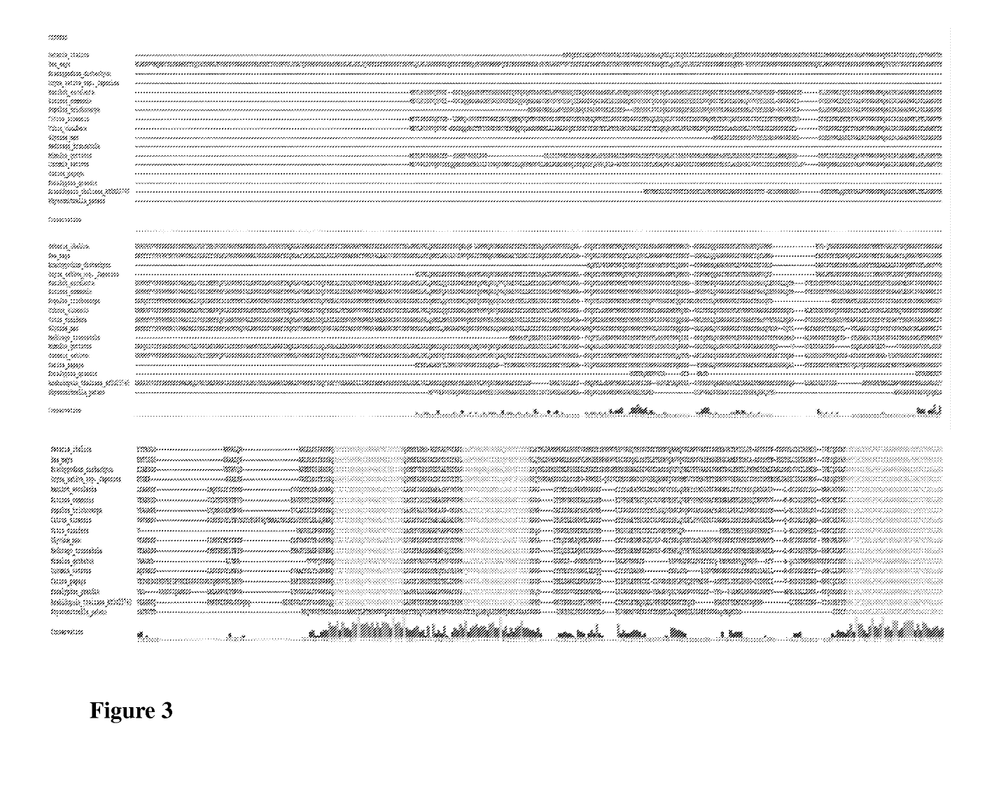

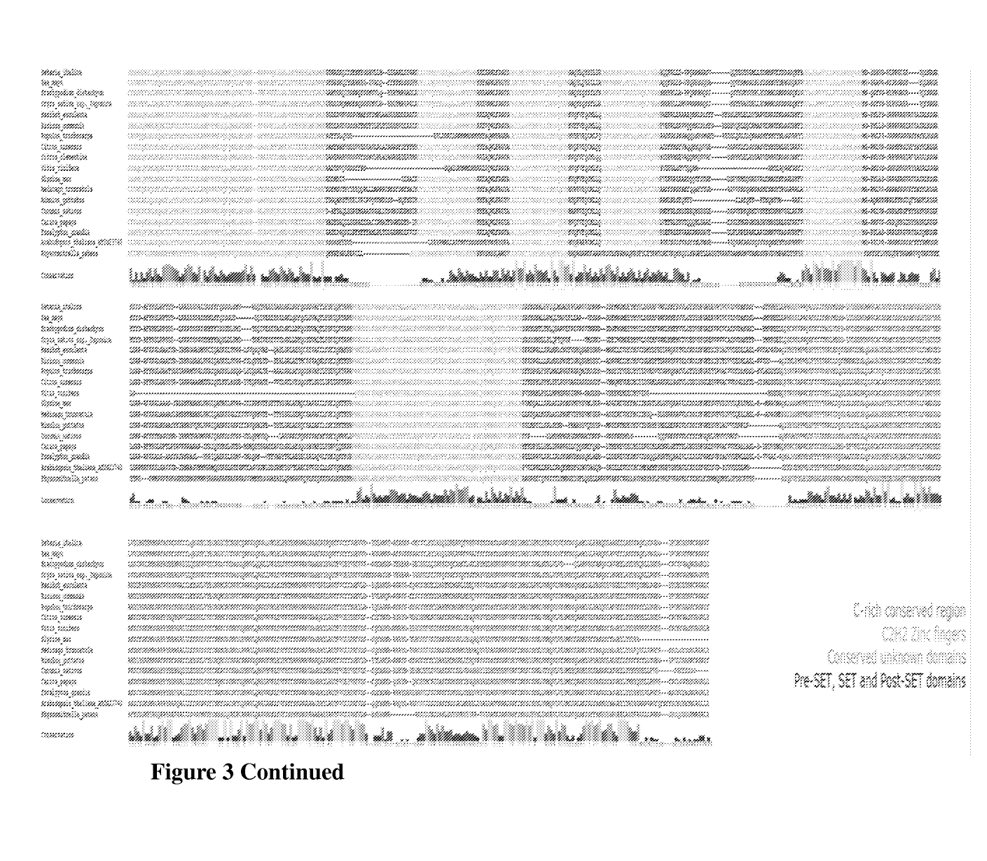

In contrast to SU(VAR)3-9 homologs, SUVR proteins lack the SRA domain that recruits the HMTase activity to chromatin. SUVR5 has three C2H2 Zinc fingers in tandem, and it may be that these zinc fingers direct the epigenetic modifier activity to specific sequence regions of the genome. While SUVR5 was conserved in all plant species analyzed, no homologs were identified in any other kingdoms (FIG. 3).

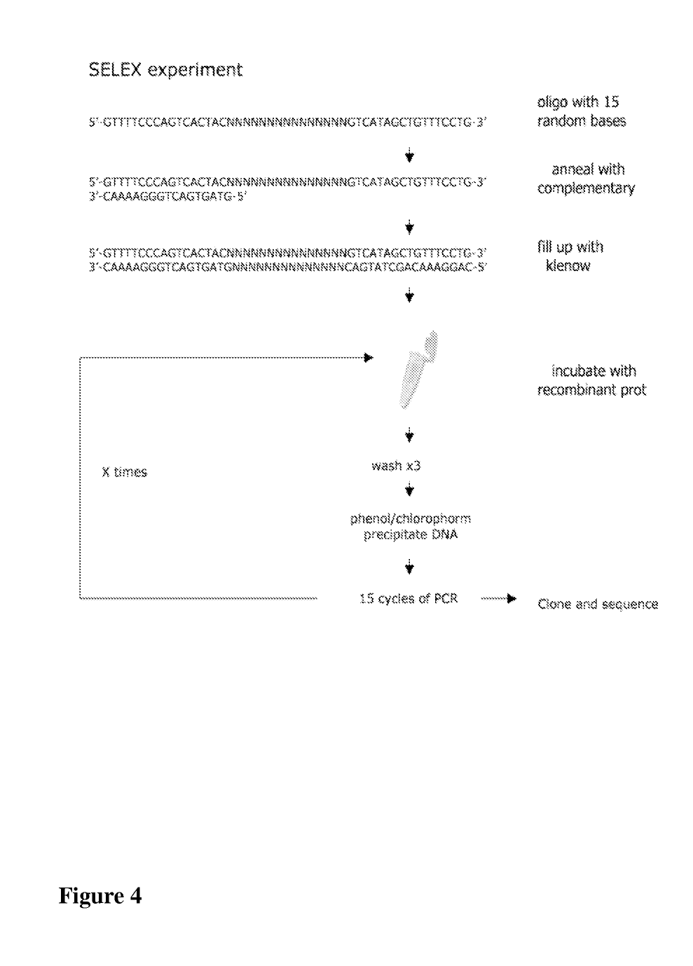

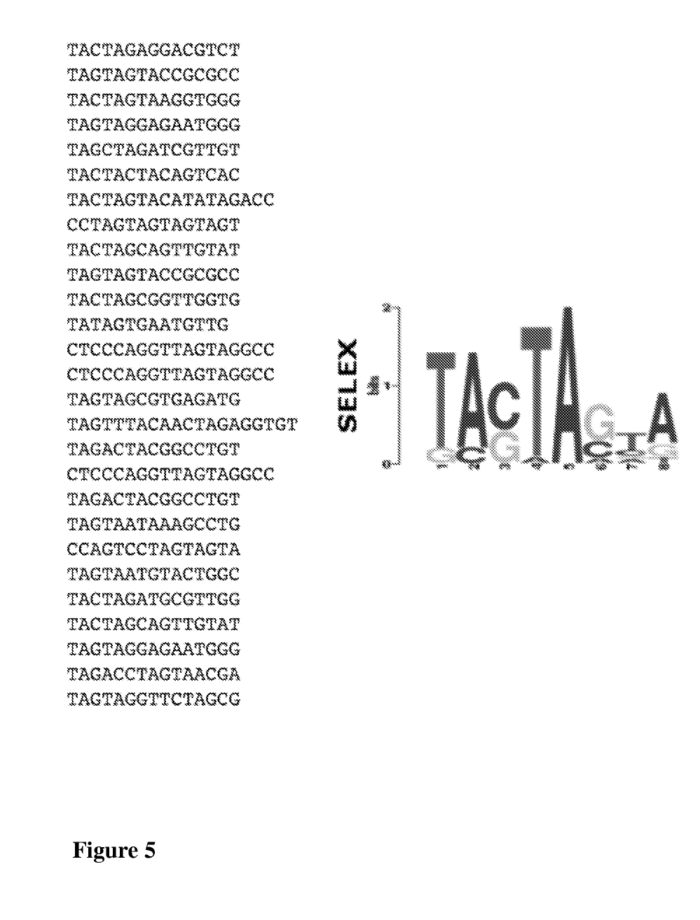

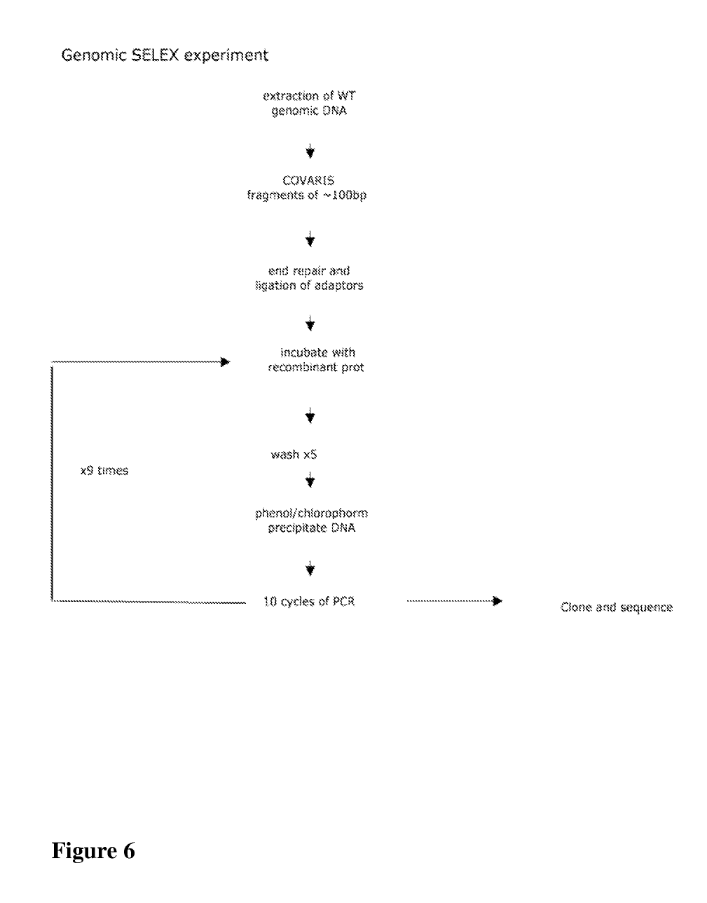

In an attempt to determine whether the zinc fingers in SUVR5 binds DNA and what specific sequence they may recognize, SELEX experiments were performed with oligos that included a 15 bp random sequence (FIGS. 4 and 5). Additionally, genomic SELEX experiments were performed with 100 bp fragments from Col0 genomic DNA (FIG. 6). Almost the same binding sequence was identified in both cases: “TACTAGTA” (FIG. 7B). The identified sequence is a palindromic octamer that fits well within the 9 nucleotide expected size of the sequence recognized by the SUVR5 zinc finger domain. The binding and specificity were also confirmed by EMSA (FIG. 7D).

The results from the genomic SELEX experiment allowed the mapping of the identified binding regions to the Arabidopsis genome, and the meta-analysis performed with the data obtained shows that the regions bound by the SUVR5 zinc finger domain map preferentially to the promoters of genes, or at least to the region upstream of the gene coding regions (FIG. 7C).

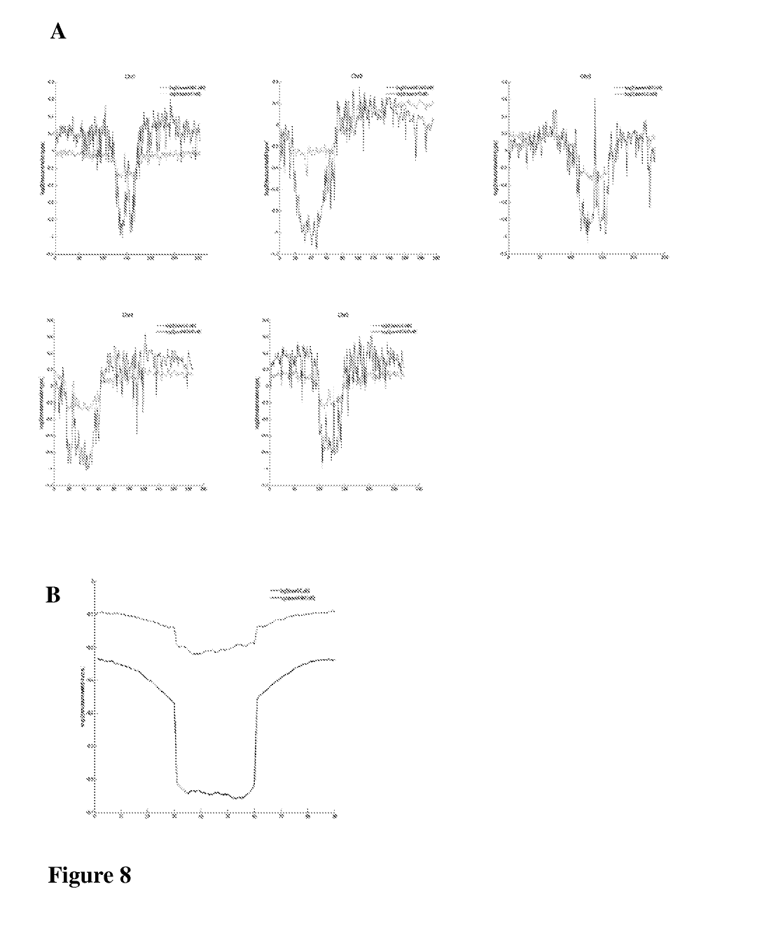

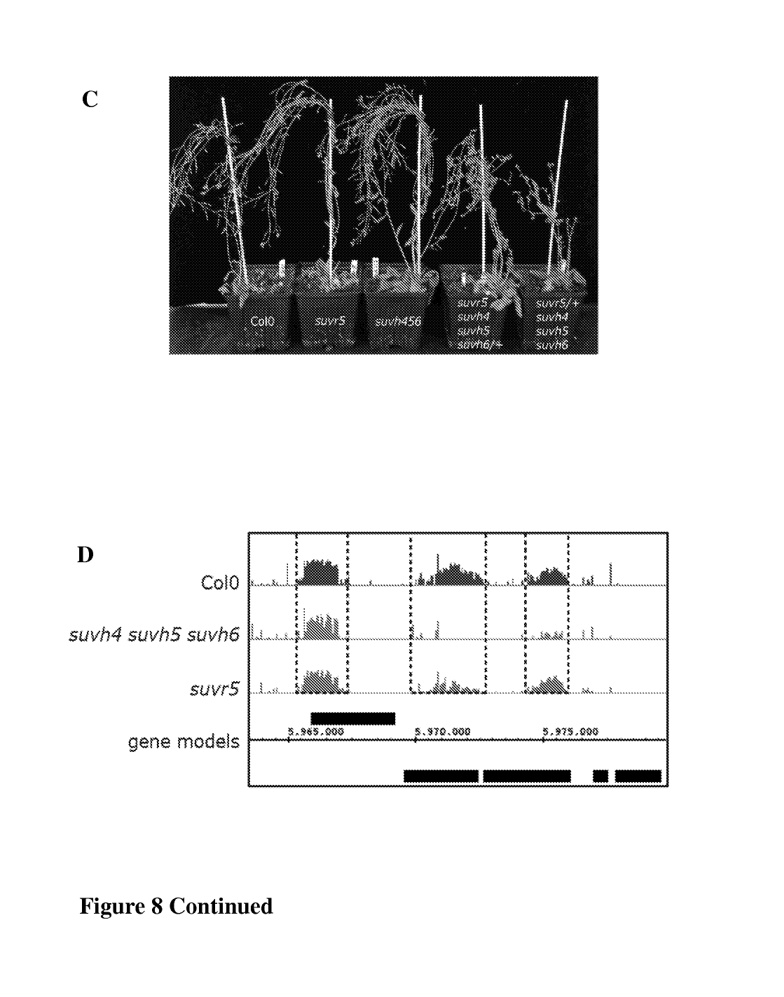

To test the function of SUVR5 as an H3K9MTase on a genome wide level, H3K9me2 ChIP-chip experiments were carried out in mature leaves of Col0 and suvr5-1 plants. The results showed a decrease in the H3K9me2 levels of heterochromatin, like pericentromeric heterochromatin and transposable elements (TEs) (FIGS. 8A and 8B). This result supports the conclusion that SUVR5 functions as an active HMTase. Heterochromatin H3K9me2 is known to be maintained by SUVH4, SUVH5, and SUVH6 [10,12,13,15,16], and the ChIP-chip data on these mutants show them to be the main factors responsible for it (FIG. 8A), but the results also indicate some level of redundancy between their functions and that of SUVR5.

The redundancy between SUVH4, SUVH5, SUVH6, and SUVR5 functions on controlling H3K9me2 deposition in pericentromeric heterochromatin and TEs is also supported by the fact that the combinations of 3 homozygous mutations and a fourth heterozygous mutation in either suvh6 or suvr5) produce developmentally challenged plants that suffer from severe infertility (FIG. 8C).

The ChIP-chip data also allowed for the verification of the redundancy of SUVH4, SUVH5, SUVH6, and SUVR5 in H3K9me2 deposition for some specific loci and to identify different kinds of heterochromatic loci, which depended on how the mutations on the HMTases affected the heterochromatic loci. FIG. 8D shows examples of adjacent loci that are not affected (left), that are affected by mutations on either the triple mutant suvh4 suvh5 suvh6 or the single mutant suvr5 (center), or that are only affected by the triple mutant suvh4 suvh5 suvh6 (right).

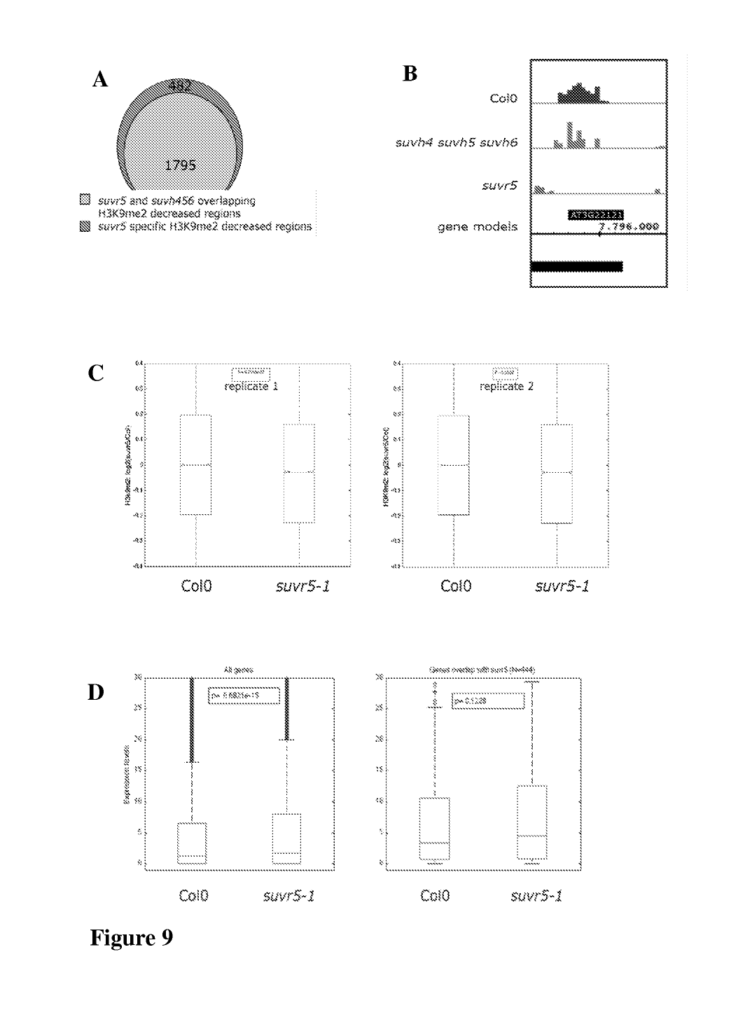

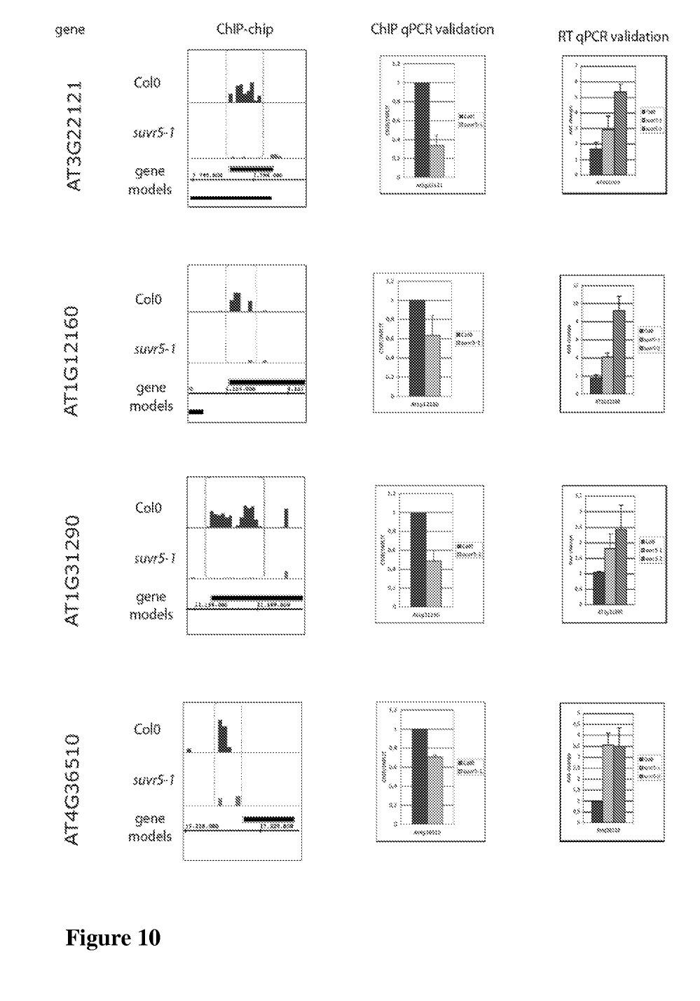

This study focused on the H3K9me2 regions dependent only on SUVR5, which accounted for 21% of the total defined regions for suvr5 mutants (FIG. 9A), and mostly mapped to the chromosome arms. An example of such a region can be seen in FIG. 9B. Additional examples together with validation data obtained by regular single locus ChIP qPCR are shown in FIG. 10.

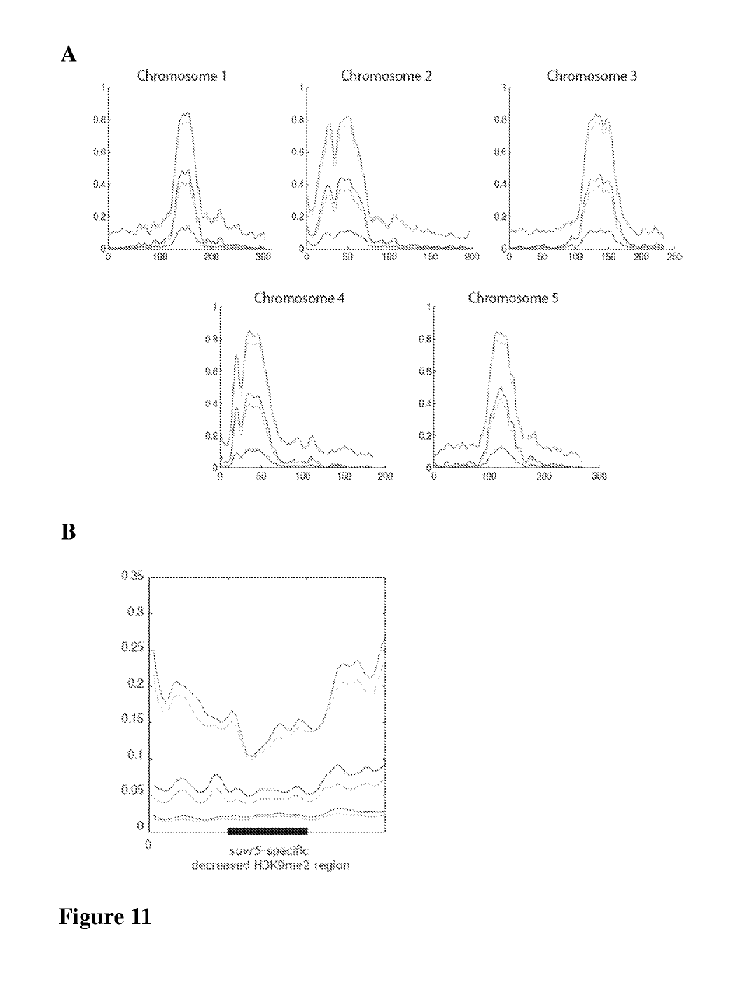

H3K9me2 has been shown to be correlated to DNA methylation in Arabidopsis in a genome wide level [17], while the loss of H3K9me2 in suvh4/kyp mutants produces a decrease in DNA methylation [10,15,16] that is enhanced in the double mutants suvh4 suvh5 and suvh4 suvh6, and the triple mutant suvh4 suvh5 suvh6 [12,13]. However, in the case of suvr5 mutants, the results did not show a decrease in any of the different types of DNA methylation accumulated in the pericentromeric heterochromatin (FIG. 11A). It may be that the loss of H3K9me2 in the single mutant is too slight to disrupt the maintenance loop established by CMT3 and KYP. When the suvr5-specific H3K9me2 decreased regions were analyzed, it was noticed that they were not characterized by high levels of DNA cytosine methylation in any context and that the little that there was, was not disturbed by the loss of SUVR5 function (FIG. 11B). These results suggest that SUVR5 is controlling H3K9me2 deposition in a DNA methylation independent manner that is not self-perpetuated by the KYP/CMT3 loop. Thus, SUVR5 may be more susceptible to changes in response to the environment or developmental cues.

Without wishing to be bound by theory, it is believed that the SUVR5 zinc finger domain may be responsible for recruiting SUVR5 to the specific locations in the chromosome arms. Moreover, despite the obvious differences between the genomic SELEX experiment (that tests binding of the recombinant protein to naked pieces of Arabidopsis DNA) and the ChIP-chip data (in vivo data obtained from actual chromatin), a correlation can still be seen that supports this zinc domain function, as the levels of H3K9me2 of the genes that show genomic SELEX signal in their promoter are significantly lower in suvr5 mutants compared to that of Col0 for both the ChIP-chip replicate experiments (FIG. 9C).

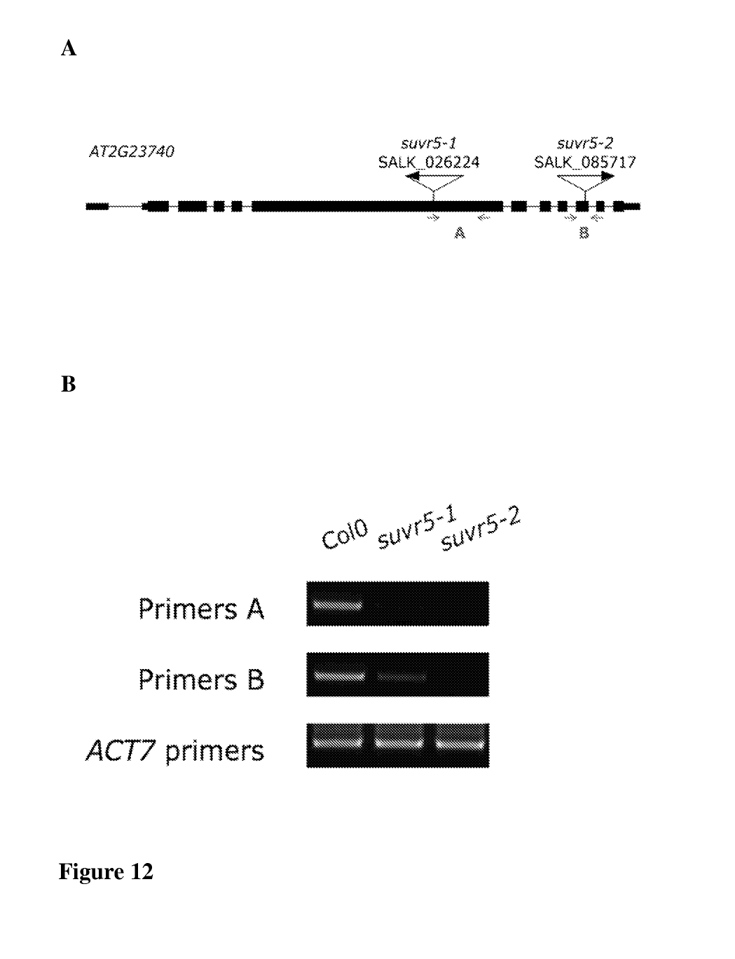

Since H3K9me2 is known to be an epigenetic repressive mark in Arabidopsis, and suvr5 mutants show a substantial decrease in H3K9me2 levels throughout the genome, it was expected, and verified by mRNAseq, that the mutant would have a global increase in gene expression. Moreover, the set of genes identified for having decreased levels of suvr5-specific H3K9me2 showed an even greater increase in gene expression in the mutants vs. Col0 than that of the average of the genome (FIG. 9D). Examples of genes that show decreased H3K9me2 levels and upregulated expression as seen by mRNA-seq and validated by RT-qPCR in two different alleles of suvr5 mutants are shown in FIG. 10. FIG. 12 shows the characterization of the two different suvr5 mutant alleles. Consistent with the decrease in H3K9me2 levels that occurs in suvr5-1 pericentromeric heterochromatin, very few transposons were reactivated in the mutants.

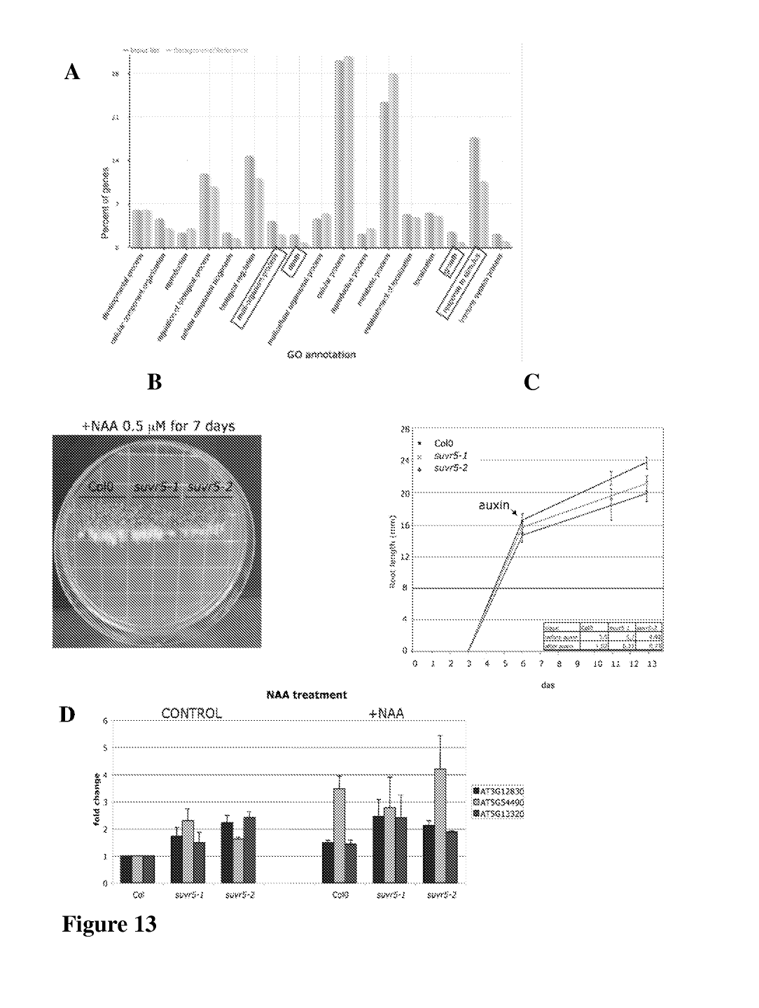

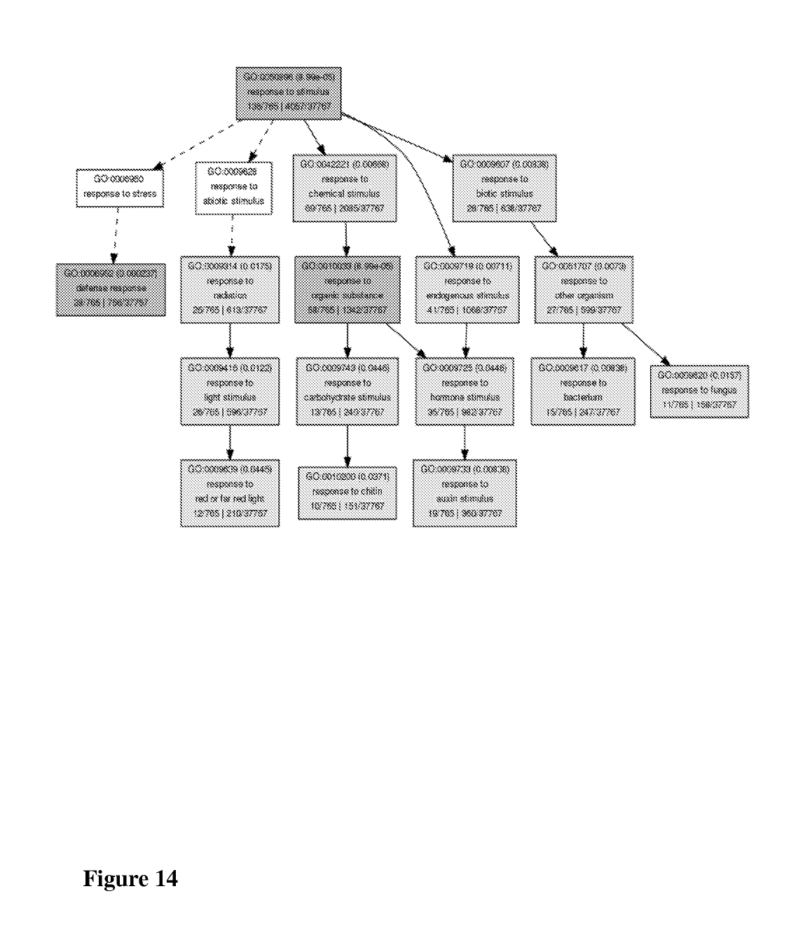

If SUVR5 is controlling H3K9me2 levels, and thus gene expression, of a certain and specific set of genes that have the identified binding motif in their promoters, then the next question to ask would be what is the biological significance of this mechanism. In order to determine the biological significance of SUVR5, a GO term analysis of the genes upregulated in the suvr5 mutant was performed. The results show that the most significant categories over-represented were the ones related with “growth” and “response to external stimulus” (FIG. 13A), including “response to auxin” (FIG. 14). When the ability of two different mutant suvr5 alleles to grow with and without auxin was tested, the results showed a delay in root lengthening of the mutants prior to hormone treatment and increased root growth inhibition after auxin application, indicating that SUVR5 has a role in controlling organ growth, and the sensitivity of the organism to hormonal signals (FIGS. 13B and 13C). These results were also supported by an expression analysis of a few examples of auxin-response genes that had been previously identified to be upregulated in the suvr5 mutant, which showed increased expression in the mutant even in the absence of the hormone (FIG. 13D). This may explain the slow growth of the mutant roots under normal conditions and the hypersensitivity to auxin (mutants have in some level already initiated the response to the stimulus even before it is actually present). These results are consistent with a model where auxin treatment overcomes the repression established by SUVR5, leaving its target genes in a state susceptible to being activated by stimuli-induced factors, and thus guaranteeing a proper response to environmental and developmental cues.

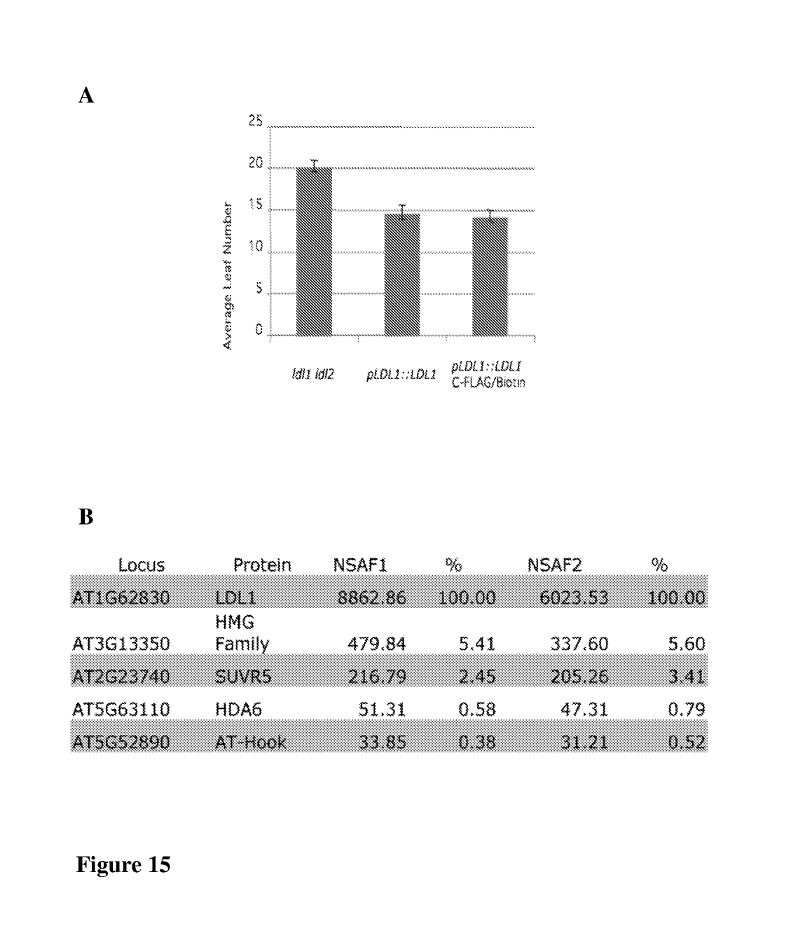

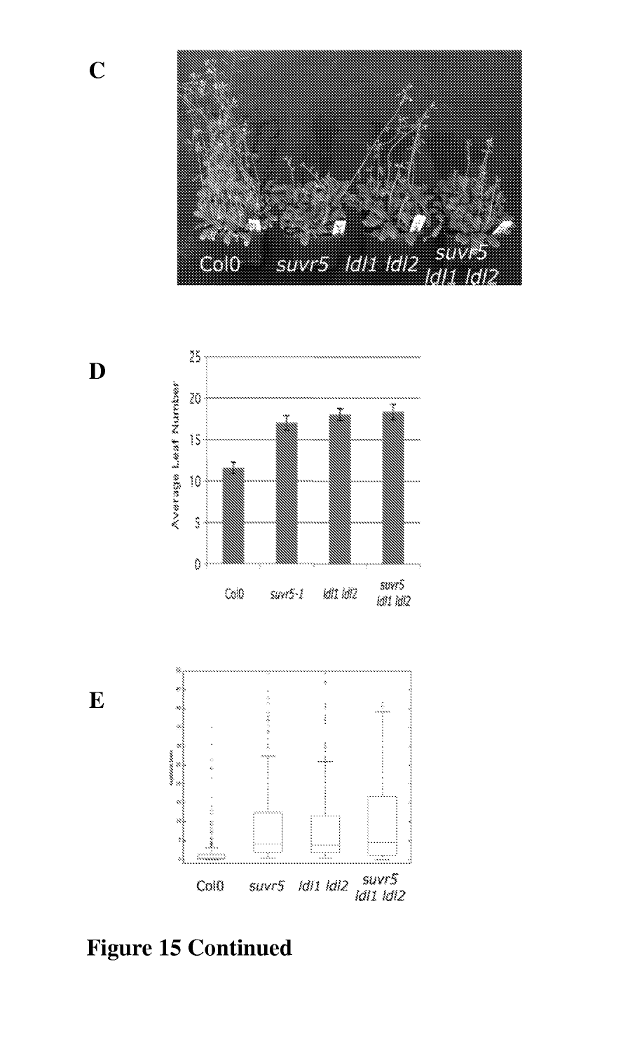

The majority of chromatin modifiers characterized in superior organisms are present in large protein multi-protein complexes. To test whether SUVR5 acts as part of a multi-protein complex in vivo, plants carrying a tagged version of the SUVR5 protein with its expression under the control of its own promoter were generated. However, efforts to pull down SUVR5 and affinity purify interactor proteins were unsuccessful. The SUVR5 protein was previously shown to interact with the LSD Arabidopsis homolog LDL1 in vitro [20]. Accordingly, a transgenic line expressing a FLAG tagged version of LDL1 under its own promoter was generated in order to determine whether SUVR5 complexes with LDL1 in vivo. The results showed that the transgenic line containing the tagged version of LDL1 was able to complement the late flowering phenotype of the ldl1 ldl2 double mutant (FIG. 15A). Thus, the protein was pulled down and the interacting proteins that accompanied it were analyzed (FIG. 15B). Two independent experiments showed the existence of an in vivo complex that includes both SUVR5 and LDL1, and the interaction between LDL1 and the histone deacetylase HDA6, suggesting that SUVR5 is part of a multimeric repressive complex, such as those that have been previously described for other higher organisms.

The genetic interaction between SUVR5 and LDL1 was then analyzed. The suvr5 ldl1 ldl2 triple mutant was generated and analyzed for flowering time. The results show an epistatic relationship between the two different mutants (FIGS. 15C and 15D).

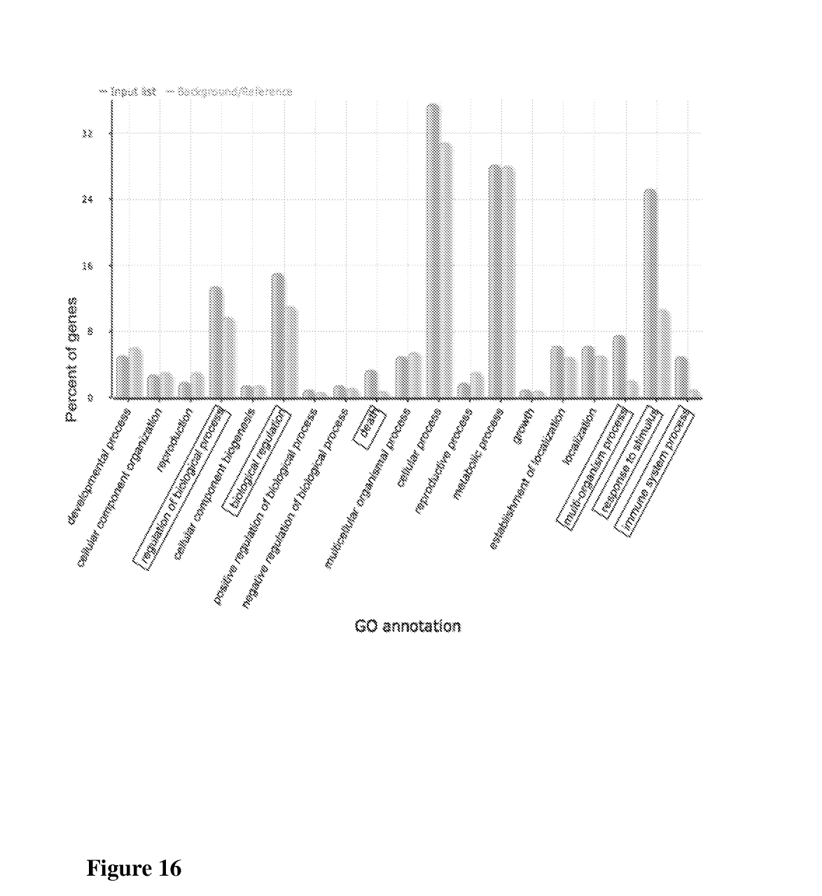

The above results confirmed the common role for SUVR5 and LDL proteins in controlling flowering time (Krichevsky et al., 2007). However, mRNAseq analysis was also performed in the suvr5 single mutant and the suvr5 ldl1 ldl2 triple mutant in order to determine how general the collaboration was between the two proteins. It was found that suvr5 and ldl1 ldl2 affect 270 genes in common, more than a 30% of the genes controlled by suvr5 alone, suggesting a more spread out common function than just the control of flowering time. It is believed that this is centered in the cellular response to a diversity of stimuli, since for the ldl1 ldl2 mutants, the GO category “response to stimulus” was also significantly enriched when clustering the upregulated genes (FIG. 16). The level of expression of the 270 common genes in Col0 was then analyzed. The expression level of the 270 genes is very low in Col0, which is consistent with being genes that are susceptible to being induced upon stimulus application. The expression level of the 270 genes was also analyzed in the suvr5 ldl1 ldl2 triple mutant. The results show that the relationship between the suvr5, ldl1, and ldl2 genes is indeed epistatic and not synergistic (FIG. 15E). This result supports the idea that SUVR5, IDL1, and IDL2 collaborate in the same pathway, with their H3K9 methylation and H3K4 demethylation activities acting together to repress gene expression for a large amount of genes with common biological functions.

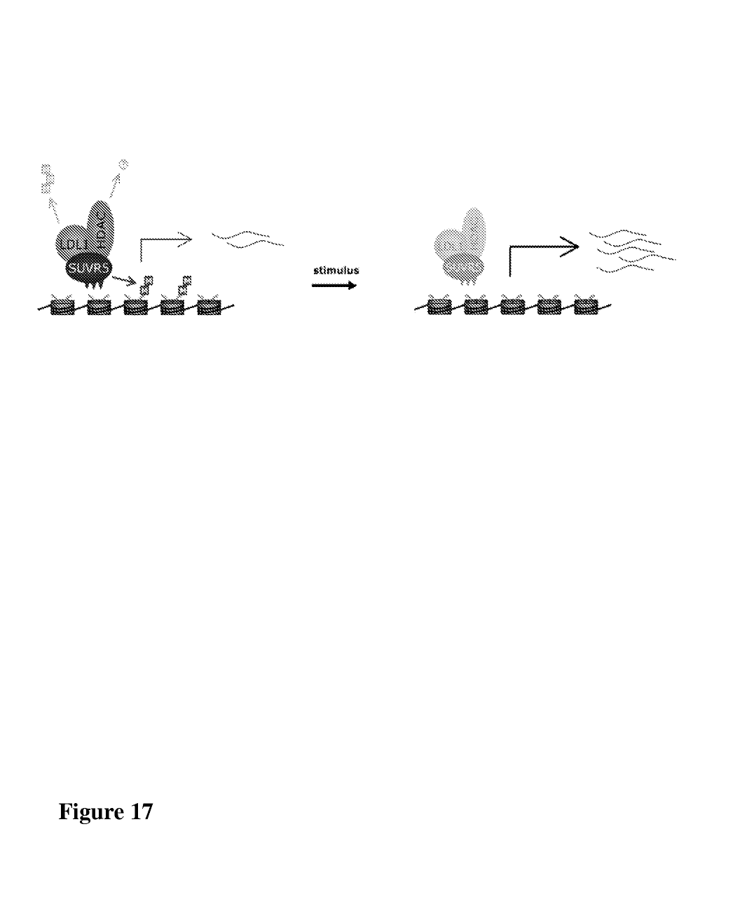

These results are consistent with a model where SUVR5 is part of a multimeric complex including LDL1 (and possibly HDA6) that recognizes genes with the sequence TACTAGTA in their promoters, and represses their expression by depositing H3K9me2 (FIG. 17).

### Discussion

The ability of eukaryotic cells to respond to external stimuli and adapt to the environment for survival depends on the coordinated activation and repression of specific subsets of genes, and to facilitate this, repressive and permissive chromatin structures must be altered in response to those stimuli. Many of the basic mechanisms regulating chromatin structure, and thus gene expression, are conserved between plants and animals, but due to the differences on the ability of these systems to respond to developmental and environmental cues, it is likely to find different strategies and mechanisms between them. The presence of a much larger family of SET domain proteins in plants may allow them a more specific control over such decisions [24]. There is a lack of information in the field about how this plasticity is achieved, about how de novo heterochromatin nucleation occurs and the involvement of factors that respond to external stimuli or developmental cues to alter chromatin states that don't necessarily need to be perpetuated generation after generation.

The results of the above Example suggest that SUVR5 maintains the heterochromatin state by H3K9me2 deposition in a DNA methylation independent way that cannot be self-perpetuated, and thus allows for changes in response to the environment or developmental cues.

The majority of chromatin modifiers characterized in animals are present in large protein multi-protein complexes. Although it is expected that some of these complexes will be conserved in plants, it is likely that many of the plant chromatin modifiers will exist in complexes that are specific to plants. The above results determined the function of AtSUVR5 as a member of one of those plant specific complexes together with the H3K4 demethylase LDL1.

The above results have identified auxin as a specific stimulus that requires SUVR activity for the plant to respond properly to the stimulus. The results also support a model in which auxin-response genes are repressed by the deposition of H3K9me2 by SUVR5 and removal of H3K4 methylation by LDL1. Without wishing to be bound by theory, it is believe that hormone stimulation overcomes the repressive state created by the SUVR5-containing protein complex, leaving these genes in a state susceptible to being activated by stimuli-induced factors, and thus guaranteeing a proper response to environmental and/or developmental cues.

Examples of complexes based on zinc finger proteins that bind to specific sequences in the genome in complex with chromatin remodelers such as histone methyl transferases, demethylases, or deacetylases have been previously described for superior organisms, but details about their function in responding to specific signals is still lacking.

The gene silencing transcription factor REST is widely expressed during embryogenesis in mammals and plays a strategic role in neural differentiation. It binds to the conserved RE1 motif through its 8 Krüppel zinc finger motifs and represses many neuronal genes in non-neuronal cells [25], in a similar way to SUVR5 mode of action [21]. This transcriptional regulation is achieved by the recruitment by REST of histone deacetylases (like HDAC1/2) [26,27,28,29], demethylases (like LSD1) [30], and methyltransferases (like G9a) [31]. The REST complex has been correlated with the molecular and cellular mechanisms that underlie the neuronal death associated with stroke, epilepsy and Huntington's disease [32,33,34].

PR (PRDI-BF1 and RIZ homology) domain proteins represent a distinct and unique branch of metazoan proteins that contain PR domain, which at the amino acid level is 20-30% identical to the SET domain found in many histone lysine methyltransferases (HMTs) [35] and that is not present in fungi or plant genomes but originates in invertebrates [36]. PR domains are almost always accompanied by C2H2-like zinc finger motifs and act as specific transcriptional regulators catalyzing histone methylation and/or recruiting interaction partners to modify the epigenetic regulation of target genes expression [35]. A common feature of PRDM proteins is their ability to act as transcriptional repressors by binding both to G9a and class I histone deacetylases enzymes as HDAC1-3 [35]. Some PRDM family members have been related to human diseases, most prominently hematological malignancies and solid cancers, where they can act as both tumor suppressors or drivers of oncogenic processes [35].

Because of the relationship of REST and PR protein complexes with disease, it is believed that the SUVR5 mechanism described for Arabidopsis thaliana provides a paradigm in a traceable system to study abnormal epigenetic regulation of gene expression in locus specific sites.

### Example 2

The following Example relates to the production and characterization of a modified A. thaliana SUVR5 protein that is engineered to replace the endogenous zinc finger domain with a heterologous zinc finger domain targeted to the FWA promoter sequence repeats. This example demonstrates the ability of the modified SUVR5 protein to induce FWA gene silencing and alter flowering time regulation in plants.

### Materials and Methods

Construct Generation

The SUVR5 coding sequence was cloned into the Gateway vector pENTR and subcloned into pGWB21 through a LR reaction for overexpression by the 35S promoter. An N-terminal 10xMyc tag was added to the recombinant sequence. For substitution of SUVR5 zinc fingers, AfeI restriction sites were introduced by site directed mutagenesis in nucleotide positions 2188 and 2581 of the pENTR-SUVR5 clone and after digestion, the 108 zinc finger domain that targets proteins to FWA promoter repeats (ZF) was introduced in these same AfeI sites. An LR reaction was performed and then ZF-SUVR5 was also cloned into pGWB21 for overexpression by the 35S promoter and an N-terminal tag of 10xMyc was included.

Plant Transformation and Transformant Selection

Fwa-4 mutant plants were transformed with both control pGWB21-SUVR5 and pGWB-ZF-SUVR5 constructs and TO plants were selected. Three transformants were selected where SUVR5 protein expression could be detected by Western Blot using an anti-Myc (6A10) antibody. Seeds were collected from these plants and grown in parallel with Col0 and fwa-4 as controls.

### Results

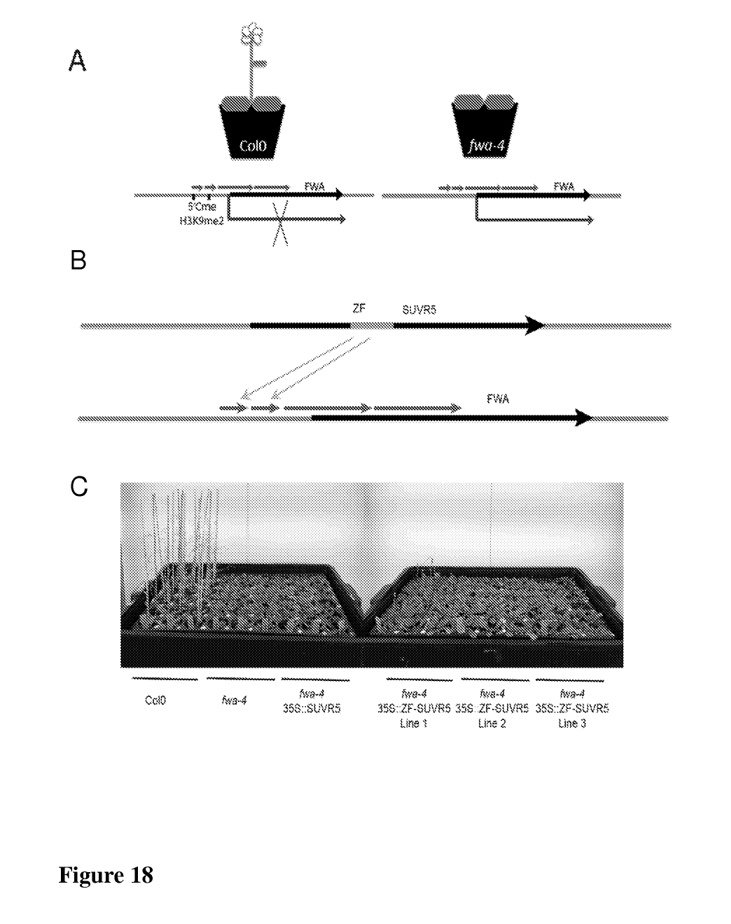

In order to demonstrate that a modified SUVR5 protein can be specifically directed to and silence a pre-selected locus, the well-studied FWA gene in Arabidopsis was utilized for this purpose. FWA can adopt two stable epigenetic states. The late flowering phenotype of fwa mutants is caused by gain-of-function epi-alleles at a homeodomain gene [43]. In most wild type Arabidopsis plants, the FWA gene is silenced by H3K9m2 and DNA methylation, which is present on two tandem repeats in the promoter of FWA (FIG. 18A). However, there are also stable epigenetic mutants of FWA, such as the fwa-4 allele, which have permanently lost this H3K9m2 and DNA methylation, causing the gene to be ectopically expressed, which causes a late flowering phenotype (FIG. 18A). Therefore, the flowering time of the plants is a direct read out of the level of expression of the FWA gene.

To test the silencing capacity of a modified SUVR5 protein, a zinc finger was designed, called the 108 zing finger (FIG. 18B), that targets the FWA repeats. The endogenous SUVR5 zinc fingers were replaced with the 108 zinc fingers (35S::ZF-SUVR5) (FIG. 18B) in order to target SUVR5 to the FWA repeats and induce silencing. FWA is ideal in this regard because it is not normally a target of SUVR5. The 35S::ZF-SUVR5 construct was transformed into fwa-4 mutants.

It was observed that fwa-4 plants transformed with the 35S::ZF-SUVR5, but not the control plants transformed with 35S::SUVR5, displayed a flowering time that was earlier than the control fwa-4 plants, indicating that the 35S::ZF-SUVR5 is causing gene silencing of the FWA gene (FIG. 18C). These results demonstrate that targeting SUVR5 to the FWA gene induced gene silencing.

### REFERENCES

1. Jenuwein T, Allis C D (2001) Translating the histone code. Science 293: 1074-1080.
2. Law J A, Jacobsen SE Establishing, maintaining and modifying DNA methylation patterns in plants and animals. Nat Rev Genet 11: 204-220.
3. Henderson I R, Jacobsen S E (2007) Epigenetic inheritance in plants. Nature 447: 418-424.
4. Woo H R, Pontes O, Pikaard C S, Richards E J (2007) VIM1, a methylcytosine-binding protein required for centromeric heterochromatinization. Genes Dev 21: 267-277.
5. Woo H R, Dittmer T A, Richards E J (2008) Three SRA-domain methylcytosine-binding proteins cooperate to maintain global CpG methylation and epigenetic silencing in Arabidopsis. PLoS Genet 4: e1000156.
6. Kraft E, Bostick M, Jacobsen S E, Callis J (2008) ORTH/VIM proteins that regulate DNA methylation are functional ubiquitin E3 ligases. Plant J 56: 704-715.
7. Johnson L M, Bostick M, Zhang X, Kraft E, Henderson I, et al. (2007) The SRA methyl-cytosine-binding domain links DNA and histone methylation. Curr Biol 17: 379-384.
8. Bostick M, Kim J K, Esteve P O, Clark A, Pradhan S, et al. (2007) UHRF1 plays a role in maintaining DNA methylation in mammalian cells. Science 317: 1760-1764.
9. Sharif J, Muto M, Takebayashi S, Suetake I, Iwamatsu A, et al. (2007) The SRA protein Np95 mediates epigenetic inheritance by recruiting Dnmt1 to methylated DNA. Nature 450: 908-912.
10. Jackson J P, Lindroth A M, Cao X, Jacobsen S E (2002) Control of CpNpG DNA methylation by the KRYPTONITE histone H3 methyltransferase. Nature 416: 556-560.
11. Malagnac F, Bartee L, Bender J (2002) An Arabidopsis SET domain protein required for maintenance but not establishment of DNA methylation. Embo J 21: 6842-6852.
12. Ebbs M L, Bender J (2006) Locus-specific control of DNA methylation by the Arabidopsis SUVH5 histone methyltransferase. Plant Cell 18: 1166-1176.
13. Ebbs M L, Bartee L, Bender J (2005) H3 lysine 9 methylation is maintained on a transcribed inverted repeat by combined action of SUVH6 and SUVH4 methyltransferases. Mol Cell Biol 25: 10507-10515.
14. Johnson L, Mollah S, Garcia B A, Muratore T L, Shabanowitz J, et al. (2004) Mass spectrometry analysis of Arabidopsis histone H3 reveals distinct combinations of post-translational modifications. Nucleic Acids Res 32: 6511-6518.
15. Jackson J P, Johnson L, Jasencakova Z, Zhang X, PerezBurgos L, et al. (2004) Dimethylation of histone H3 lysine 9 is a critical mark for DNA methylation and gene silencing in Arabidopsis thaliana. Chromosoma 112: 308-315.
16. Tariq M, Saze H, Probst A V, Lichota J, Habu Y, et al. (2003) Erasure of CpG methylation in Arabidopsis alters patterns of histone H3 methylation in heterochromatin. Proc Natl Acad Sci USA 100: 8823-8827.
17. Bernatavichute Y V, Zhang X, Cokus S, Pellegrini M, Jacobsen S E (2008) Genome-wide association of histone H3 lysine nine methylation with CHG DNA methylation in Arabidopsis thaliana. PLoS One 3: e3156.
18. Rajakumara E, Law J A, Simanshu D K, Voigt P, Johnson L M, et al. A dual flip-out mechanism for 5mC recognition by the Arabidopsis SUVH5 SRA domain and its impact on DNA methylation and H3K9 dimethylation in vivo. Genes Dev 25: 137-152.
19. Law J A, Ausin I, Johnson L M, Vashisht A A, Zhu J K, et al. A protein complex required for polymerase V transcripts and RNA-directed DNA methylation in Arabidopsis. Curr Biol 20: 951-956.
20. Krichevsky A, Gutgarts H, Kozlovsky S V, Tzfira T, Sutton A, et al. (2007) C2H2 zinc finger-SET histone methyltransferase is a plant-specific chromatin modifier. Dev Biol 303: 259-269.
21. Krichevsky A, Kozlovsky S V, Gutgarts H, Citovsky V (2007) Arabidopsis co-repressor complexes containing polyamine oxidase-like proteins and plant-specific histone methyltransferases. Plant Signal Behav 2: 174-177.
22. Baumbusch L O, Thorstensen T, Krauss V, Fischer A, Naumann K, et al. (2001) The Arabidopsis thaliana genome contains at least 29 active genes encoding SET domain proteins that can be assigned to four evolutionarily conserved classes. Nucleic Acids Res 29: 4319-4333.
23. Mylne J S, Barrett L, Tessadori F, Mesnage S, Johnson L, et al. (2006) LHP1, the Arabidopsis homologue of HETEROCHROMATIN PROTEIN1, is required for epigenetic silencing of FLC. Proc Natl Acad Sci USA 103: 5012-5017.
24. Springer N M, Napoli C A, Selinger D A, Pandey R, Cone K C, et al. (2003) Comparative analysis of SET domain proteins in maize and Arabidopsis reveals multiple duplications preceding the divergence of monocots and dicots. Plant Physiol 132: 907-925.
25. Schoenherr C J, Paquette A J, Anderson D J (1996) Identification of potential target genes for the neuron-restrictive silencer factor. Proc Natl Acad Sci USA 93: 9881-9886.
26. Grimes J A, Nielsen S J, Battaglioli E, Miska E A, Speh J C, et al. (2000) The co-repressor mSin3A is a functional component of the REST-CoREST repressor complex. J Biol Chem 275: 9461-9467.
27. Huang Y, Myers S J, Dingledine R (1999) Transcriptional repression by REST: recruitment of Sin3A and histone deacetylase to neuronal genes. Nat Neurosci 2: 867-872.
28. Naruse Y, Aoki T, Kojima T, Mori N (1999) Neural restrictive silencer factor recruits mSin3 and histone deacetylase complex to repress neuron-specific target genes. Proc Natl Acad Sci USA 96: 13691-13696.
29. Roopra A, Sharling L, Wood I C, Briggs T, Bachfischer U, et al. (2000) Transcriptional repression by neuron-restrictive silencer factor is mediated via the Sin3-histone deacetylase complex. Mol Cell Biol 20: 2147-2157.
30. Shi Y, Lan F, Matson C, Mulligan P, Whetstine J R, et al. (2004) Histone demethylation mediated by the nuclear amine oxidase homolog LSD1. Cell 119: 941-953.
31. Tachibana M, Sugimoto K, Fukushima T, Shinkai Y (2001) Set domain-containing protein, G9a, is a novel lysine-preferring mammalian histone methyltransferase with hyperactivity and specific selectivity to lysines 9 and 27 of histone H3. J Biol Chem 276: 25309-25317.
32. Ooi L, Wood I C (2007) Chromatin crosstalk in development and disease: lessons from REST. Nat Rev Genet 8: 544-554.
33. Buckley N J, Johnson R, Zuccato C, Bithell A, Cattaneo E The role of REST in transcriptional and epigenetic dysregulation in Huntington's disease. Neurobiol Dis 39: 28-39.
34. Gillies S, Haddley K, Vasiliou S, Bubb V J, Quinn J P (2009) The human neurokinin B gene, TAC3, and its promoter are regulated by Neuron Restrictive Silencing Factor (NRSF) transcription factor family. Neuropeptides 43: 333-340.
35. Fog C K, Galli G G, Lund A H PRDM proteins: Important players in differentiation and disease. Bioessays.
36. Kim K C, Huang S (2003) Histone methyltransferases in tumor suppression. Cancer Biol Ther 2: 491-499.
37. Grewal S I, Moazed D (2003) Heterochromatin and epigenetic control of gene expression. Science 301: 798-802.
38. Jiang D, Yang W, He Y, Amasino R M (2007) Arabidopsis relatives of the human lysine-specific Demethylasel repress the expression of FWA and FLOWERING LOCUS C and thus promote the floral transition. Plant Cell 19: 2975-2987.
39. Johnson L, Cao X, Jacobsen S (2002) Interplay between two epigenetic marks. DNA methylation and histone H3 lysine 9 methylation. Curr Biol 12: 1360-1367.
40. Li C F, Pontes O, El-Shami M, Henderson I R, Bernatavichute Y V, et al. (2006) An ARGONAUTE4-containing nuclear processing center colocalized with Cajal bodies in Arabidopsis thaliana. Cell 126: 93-106.
41. Sasai N, Nakao M, Defossez P A Sequence-specific recognition of methylated DNA by human zinc-finger proteins. Nucleic Acids Res 38: 5015-5022.
42. Bailey T L, Boden M, Buske F A, Frith M, Grant C E, et al. (2009) MEME SUITE: tools for motif discovery and searching. Nucleic Acids Res 37: W202-208.
43. Soppe W J, Jacobsen S E, Alonso-Blanco C, Jackson J, Kakutani T, Koornneef M, Peeters AJM. (2000) The late flowering phenotype of fwa mutants is caused by gain of function epi-alleles at a homeodomain gene. Molecular Cell 6: 791-802.

1. A method for reducing expression of one or more target nucleic acids in a plant, comprising:
(a) providing a plant comprising a recombinant polypeptide, wherein the recombinant polypeptide comprises a DNA-binding domain, a C-terminal pre-SET domain, a C-terminal SET domain, and a C-terminal post-SET domain; and
(b) growing the plant under conditions whereby the recombinant polypeptide binds to the one or more target nucleic acids, thereby reducing expression of the one or more target nucleic acids.

2. The method of claim 1, wherein the DNA-binding domain comprises a zinc finger domain.

3. (canceled)

4. (canceled)

5. (canceled)

6. (canceled)

7. (canceled)

8. The method of claim 2, wherein the DNA-binding domain comprises three C2H2 zinc finger domains.

9. The method of claim 1, wherein the DNA-binding domain comprises an amino acid sequence that is at least 80% identical to SEQ ID NO: 1.

10. (canceled)

11. The method of claim 1, wherein the recombinant polypeptide further comprises one or more additional DNA-binding domains.

12. The method of claim 11, wherein the one or more additional DNA-binding domains comprise an amino acid sequence that is at least 80% identical to SEQ ID NO: 1.

13. (canceled)

14. The method of claim 11, wherein the one or more additional DNA-binding domains comprise an amino acid sequence heterologous to SEQ ID NO: 1.

15. The method of claim 1, wherein the C-terminal pre-SET domain comprises an amino acid sequence that is at least 80% identical to SEQ ID NO: 2 or SEQ ID NO: 3.

16. The method of claim 1, wherein the C-terminal SET domain comprises an amino acid sequence that is at least 80% identical to SEQ ID NO: 4, SEQ ID NO: 5, or SEQ ID NO: 6.

17. The method of claim 1, wherein the C-terminal post-SET domain comprises an amino acid sequence that is at least 80% identical to SEQ ID NO: 7.

18. The method of claim 1, wherein the recombinant polypeptide comprises an amino acid sequence that is at least 80% identical to SEQ ID NO: 8.

19. (canceled)

20. (canceled)

21. (canceled)

22. (canceled)

23. (canceled)

24. (canceled)

25. (canceled)

26. (canceled)

27. (canceled)

28. (canceled)

29. The method of claim 1, wherein the one or more target nucleic acids are endogenous nucleic acids.

30. The method of claim 1, wherein the one or more target nucleic acids are transgenes.

31. The method of claim 1, wherein expression of the one or more target nucleic acids is silenced.

32. A recombinant nucleic acid encoding an SUVR5-like protein, wherein the SUVR5-like protein comprises a DNA-binding domain, a C-terminal pre-SET domain, a C-terminal SET domain, and a C-terminal post-SET domain; and wherein the DNA-binding domain is heterologous to an SUVR5 DNA-binding domain.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. A method for reducing expression of one or more target nucleic acids in a plant, comprising:
a) providing a plant comprising a recombinant polypeptide, wherein the recombinant polypeptide comprises a DNA-binding domain, a C-terminal pre-SET domain, a C-terminal SET domain, and a C-terminal post-SET domain; and
b) growing the plant under conditions whereby the recombinant polypeptide is targeted to the one or more nucleic acids, thereby reducing expression of the one or more target nucleic acids.

72. The method of claim 71, wherein the C-terminal pre-SET domain comprises an amino acid sequence that is at least 80% identical to SEQ ID NO: 2 or SEQ ID NO: 3.

73. The method of claim 71, wherein the C-terminal SET domain comprises an amino acid sequence that is at least 80% identical to SEQ ID NO: 4, SEQ ID NO: 5, or SEQ ID NO: 6.

74. The method of claim 71, wherein the C-terminal post-SET domain comprises an amino acid sequence that is at least 80% identical to SEQ ID NO: 7.

75. The method of claim 71, wherein the one or more target nucleic acids are endogenous nucleic acids.
